(19) United States
(12) Patent Application Publication
(10) Pub. No.: US 2009/0098164 A1
(43) Pub. Date: Apr. 16, 2009

(54) Spore associated display

(76) Inventors: Ramesh Bhatt, Belmont, CA (US); Lawrence Horowitz, Atherton, CA (US); Angeles Estelles, Belmont, CA (US)

Correspondence Address: Goodwin Procter LLP;Attn: Patent Administrator, 135 Commonwealth Drive, Menlo Park, CA, 94025-1105, US

(21) Appl. No.: 12/228,655

(22) Filed: Aug. 13, 2008

Related U.S. Application Data
(60) Provisional application No. 60/995,967, filed on Sep. 28, 2007.
(60) Provisional application No. 60/955,592, filed on Aug. 13, 2007.

(51) Int. Cl. A61K 39/07; C07K 2/00; C07K 14/00; C07K 16/12; C12N 9/96; C07H 21/04; C12N 1/21; C40B 40/10; C40B 40/06; C12N 15/74

(52) U.S. Cl. 424/246.1; 530/300; 530/350; 530/387.3; 435/188; 536/23.4; 536/23.71; 435/252.3; 506/18; 506/16; 435/471

(57) ABSTRACT

The present invention concerns spore display methods. More specifically, the invention concerns display of heterologous molecules, such as peptides and polypeptides, on spores of bacilli, such as, for example, Bacillus thuringiensis (Bt) or Bacillus cereus (BC), using externally exposed spore coat proteins or fragments or variants thereof.

### CROSS REFERENCE TO RELATED APPLICATIONS

This application is a non-provisional application filed under 37 CFR 1.53(b)(1), claiming priority under 35 USC 119(e) to provisional application No. 60/995,967 filed Sep. 28, 2007, and provisional application No. 60/955,592 filed Aug. 13, 2007, the contents of which are incorporated herein by reference.

### FIELD OF THE INVENTION

The present invention concerns spore display methods. More specifically, the invention concerns display of heterologous molecules, such as peptides and polypeptides, on spores of bacilli, such as, for example, Bacillus thuringiensis (Bt) or Bacillus cereus (BC), using externally exposed spore coat proteins or fragments or variants thereof.

### BACKGROUND OF THE INVENTION

There are systems known in the art for display of heterologous proteins on the surfaces of bacteriophage (Scott et al., Science 249:386-390 (1990)), Escherichia coli (Agterberg et al., Gene 88:37-45 (1990); Charbit et al., Gene 70:181-189 (1988); Francisco et al., Proc. Natl. Acad. Sci. USA 89:2713-2717 (1992)) and Saccharomyces cerevisiae (Boder and Wittrup, Nat. Biolechnol. 15:553-557 (1997)). In phage display, the heterologous protein, such as a single-chain antibody fragment (scFv), is linked to a coat protein of a phage particle, while the DNA sequence from which it was expressed is packaged within the phage coat. A display and selection system (referred to as APEx for “Anchored Periplasmic Expression”) based on anchoring proteins, such as single-chain antibody fragments, to the periplasmic face of the inner membrane of Escherichia coli has also been developed.

Display of heterologous proteins Bacillus spores has also been described. The spore protoplast (core) is surrounded by the cell wall, the cortex, and the spore coat. Depending on the species, an exosporium may also be present. The core wall is composed of the same type of peptidoglycan as the vegetative cell wall. A surface display system using a component of the Bacillus subtilis spore coat (CotB), has been described by Isticato et al., J. Bacteriol. 183:6294-6301 (2001).

Bacillus thuringiensis (Bt) is a rod-shaped, Gram-positive bacterium, which has been extensively studied, primarily due to its importance in insect control. Bt produces a large number of proteins that are toxic to insects. Bt also produces several enzymes, compounds that lyse erythrocytes, and compounds that are enterotoxic to vertebrates. Bt toxins are produced either within the bacterial cell (endotoxins), or on the cell surface (exotoxins). Bt is distinguished from the closely related Bacillus cereus and Bacillus anthracis by the presence of several plasmid-encoded delta-endotoxin genes. These delta-endotoxins, synthesized as protoxins, are produced in large quantities during sporulation and are packaged into intracellular inclusions. The paracrystalline inclusions are comprised of 130- to 140-kDa delta endotoxin polypeptides, which are the predominant parasporal component of most Bt species. It has been reported that Bt protoxins are also a major component of the spore coat (Du et al., Appl. Environ. Microbiol. 71(6):3337-3341 (2005)), but they are not spore coat proteins. For example, the 130-kDa Bt protoxin from the Cry1Ac subgroup is a major component of the spore coat, and it has been proposed that the N-terminal end of the Bt protoxin is exposed on the spore surface and the C-terminal region anchors the protoxin inside the spore coat (Du and Nickerson, Appln. Eviron. Microbiol. 62:3722-3926 (1996). Cheng et al., Appl. Environ. Microbiol. 71:3337-3341 (2005) reported a display system in which the N-terminal portion of the Bt protoxin is replaced with nucleic acid encoding the green fluorescent protein (GFP) or a single-chain antibody (scFv). The authors have shown that their protoxin-scFv fusion binds fluorescently-labeled antigen and competes with unlabeled antigen, as measured by immunofluorescence.

### SUMMARY OF THE INVENTION

The present invention concerns surface display systems based on conjugates of heterologous molecules to externally exposed spore coat protein sequences. In particular, the invention concerns conjugates, such as fusion proteins, comprising the fusions of heterologous peptides or polypeptides, such as recombinant polypeptides, to the N- and/or C-terminus of spore coat protein B's (CotB proteins) of bacilli.

Thus, in one aspect, the invention concerns a conjugate comprising:

(a) the full-length sequence of an externally exposed native sequence spore coat protein of a Bacillus; or

(b) a functional fragment of an externally exposed native sequence spore coat protein of a Bacillus, other than Bacillus sublilis, or

(c) a functional variant of (a) or (b),

conjugated to a heterologous molecule.

In one embodiment, the conjugate is displayed on the surface of a Bacillus spore, where the heterologous molecule may, for example, be a peptide, a polypeptide, or a non-peptide small organic molecule.

In other embodiments, the heterologous molecule is an antibody or an antibody fragment, or a surrobody or a surrobody fragment, where the antibody fragment may, for example, be an antibody heavy or light chain, or a fragment thereof.

In yet another embodiment, the conjugate is a direct fusion between the spore coat protein and the heterologous molecule, where the fusion may be at the C- or N-terminus of the spore coat protein.

In a preferred embodiment, the fusion is at the N-terminal end of the spore coat protein.

In another embodiment, the heterologous molecule is linked to the coat protein through a linker.

In one more specific embodiment, the linker is a peptide sequence. In a preferred embodiment, the fusion is through a toxin-based short N-terminal peptide, which precedes the mature N-terminus of the spore coat protein. In another preferred embodiment, the toxin-based short N-terminal peptide is part of the native Bacillus delta protoxin from which the spore coat protein originates, or a fragment or variant thereof.

In another embodiment, the peptide linker sequence comprises a substrate sequence for an enzyme, wherein the enzyme may, for example, be a protease.

In yet another embodiment, the linker is a dimeric linker, which may comprise a covalent association between two binding partners, such as a covalent association provided by a disulfide bond. In another embodiment the dimeric linker may comprise a non-covalent association between two partners, such as, for example, between a pair of leucine zipper peptides.

In all embodiments, the Bacillus may be any spore forming Bacillus including, without limitation, Bacillus thuringiensis, Bacillus cereus, Bacillus anthracis, Bacillus amyloliquefaciens, Bacillus weihenstephanensis; Geobacillus kaustophilus; and Geobacillus thermodenitrificans.

In a preferred embodiment, the Bacillus is Bacillus thuringiensis.

In another preferred embodiment, the conjugates of the present invention comprise Bacillus thuringiensis CotB1 (SEQ ID NO: 6) or CotB2 (SEQ ID NO: 7), or a functional fragment or variant thereof.

In yet another embodiment, in the conjugates of the present invention the functional variant is a chimeric molecule comprising externally exposed spore coat protein sequences from more than one Bacilli, or more than species or sub-species of the same Bacillus, wherein in various embodiments, at least one of said Bacilli is Bacillus thuringiensis, or at least one of said Bacilli is Bacillus subtilis.

In another aspect, the invention concerns a nucleic acid molecule comprising a nucleotide sequence encoding the conjugates of the present invention, as hereinabove defined.

The encoding nucleic acid may additionally comprise regulatory sequences capable of directing the expression of the nucleic acid molecule on a Bacillus spore, where the regulatory sequences may, for example, comprise a sporulation-specific promoter region.

In another embodiment, the nucleic acid molecule comprises a nucleotide sequence encoding an N-terminal Bacillus peptide preceding the coding sequence of the mature native sequence spore coat protein or a functional fragment or variant thereof.

In a further aspect, the invention concerns a cell of a spore forming Bacillus comprising and capable of expressing the nucleic acid molecule described above.

In a still further aspect, the invention concerns a recombinant sporulating spore forming Bacillus expressing the conjugates of the present invention on the surface of the spores thereof.

In various embodiments, the Bacillus is selected from the group consisting of Bacillus thuringiensis, Bacillus cereus, Bacillus anthracis, Bacillus amyloliquefaciens, Bacillus weihenstephanensis; Geobacillus kaustophilus; and Geobacillus thermodenitrificans, and preferably is Bacillus thuringiensis.

In yet another aspect, the invention concerns a cell culture comprising cells of a recombinant sporulating spore forming Bacillus expressing the conjugates of the present invention on the surface of the spores thereof.

In a different aspect, the invention concerns a display system comprising a plurality of conjugates comprising:

(a) the full-length sequence of an externally exposed native sequence spore coat protein of a Bacillus; or

(b) a functional fragment of an externally exposed native sequence spore coat protein of a Bacillus, other than Bacillus subtilis, or

(c) a functional variant of (a) or (b),

conjugated to one or more heterologous molecules.

In one embodiment, the heterologous molecules present in the conjugates are peptides or polypeptides.

In another embodiment, the peptides or polypeptides are members of a peptide or polypeptide library.

In yet another embodiment, the peptides or polypeptides are structurally and/or functionally related to each other.

In other embodiments, the polypeptides are antibodies or antibody fragments, or surrobodies or surrobody fragments, where the antibody fragments may, for example, be selected from the group consisting of Fab, Fab′, F(ab′)₂, scFv, (scFv)₂, dAb, complementarity determining region (CDR) fragments, linear antibodies, single-chain antibody molecules, minibodies, diabodies, and multispecific antibodies formed from antibody fragments.

In a further embodiment, the heterologous molecules are non-peptide small molecules.

In still further embodiments the conjugates comprise direct fusions between the spore coat protein and the heterologous molecules, or comprise the spore coat protein and the heterologous molecules linked through a heterologous linker. Various exemplary embodiments of direct fusions and heterologous linkers are the same as those listed above.

In another embodiment, in the display system of the present invention at least some of the conjugates comprise multiple copies of the sequence of the coat protein, where each of the conjugates may comprise the same spore coat protein sequence, or at least some of the conjugates comprise different spore coat protein sequences.

In a further embodiment, the conjugates comprise monomeric units of a multimeric polypeptide.

In a still further embodiment, the monomeric units are displayed in a proximity that allows combination of said units to form a multimeric polypeptide, where the multimeric polypeptide may be, without limitation, a dimeric, trimeric, tetrameric, etc. polypeptide.

In various embodiments, the multimeric polypeptide is or comprises an antibody or antibody fragment and the monomeric units displayed are antibody heavy and light chains or fragments thereof.

In additional embodiments, the multimeric polypeptide is or comprises a surrobody or surrobody fragment.

In a different embodiment, in the display systems of the present invention the spores are bar-coded to provide unique labels, where the unique label may, for example, be a nucleic acid barcode generated by combinations of three to 20 nucleotides.

In another aspect, the invention concerns a method for displaying a collection of peptide or polypeptides on the surface of spores, comprising expressing said collection of peptides or polypeptides on the surface of spores of a Bacillus in the form of conjugates comprising:

(a) the full-length sequence of an externally exposed native sequence spore coat protein of a Bacillus; or

(b) a functional fragment of an externally exposed native sequence spore coat protein of a Bacillus, other than Bacillus subtilis, or

(c) a functional variant of (a) or (b),

conjugated to said peptides or polypeptides.

In one embodiment, substantially all of the spores are exosporium-free.

In another embodiment, at least about 90% of the spores are exosporium-free.

In yet another embodiment, the Bt spores are previously selected to be exosporium-free mutants.

In a further embodiment, the Bacillus is Bacillus thuringiensis.

In a still further embodiment, the displayed conjugates are formed by transforming Bacillus with nucleic acid encoding the conjugates, each under control of a sporulation specific promoter, and culturing and harvesting the transformed Bacillus under conditions to support sporulation and stable protein display.

In a more specific embodiment, colonies of the transformed spores are grown in a sporulation medium for less than 48 hours, such as, for example, for about 14 to about 20 hours, whereupon the spores are liberated retaining the majority of the displayed peptides or polypeptides in an intact, non-degraded form.

In other embodiments, the method further comprises a step of testing the stability of the display and/or testing the chemical or biological integrity of one or more peptides or polypeptides displayed and/or selecting the Bacillus spores displaying a coat protein-peptide or coat protein-polypeptide conjugate.

In yet another embodiment, the selection is performed by magnetic sorting and/or by flow cytometry.

In a further aspect, the invention concerns a spore carrying a fusion polypeptide of the present invention.

In one embodiment, the fusion polypeptide is stably anchored to the spore.

In another embodiment, the heterologous peptide or polypeptide is displayed on the surface of the spore.

In yet another embodiment, the heterologous peptide or polypeptide is biologically active.

In a further embodiment, the heterologous peptide or polypeptide is a therapeutic agent.

In a further aspect, the invention concerns a vaccine comprising an antigen-Bacillus coat protein conjugate displayed on the surface of a spore.

In various representative embodiments, the vaccine may be suitable for oral administration, or for transmucosal delivery, or for parenteral administration, wherein transmucosal delivery may, for example be intra-nasal administration.

The vaccine can be any kind of vaccine, including, without limitation, flu vaccines, vaccines for childhood immunization, HIV vaccines.

These and further embodiments will be apparent from the entirety of the specification, including the examples and the appended claims.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fees.

FIG. 1 shows a nucleotide sequence encoding the CotB1 spore coat protein of Bt (SEQ ID NO: 1).

FIG. 2 shows a nucleotide sequence encoding the CotB2 spore coat protein of Bt (SEQ ID NO: 2).

FIG. 3 shows the sequence of the p5-CotB1-GFP vector (SEQ ID NO: 3).

FIG. 4 is a graphic illustration of the p5-CotB1-GFP vector construct.

FIG. 5 (SEQ ID NO: 4) is the nucleotide sequence of the tetanus toxin fragment C (TTFC).

FIG. 6 (SEQ ID NO: 5) is the nucleotide sequence of an scFv construct of the anti-human TNF-α antibody D2E7.

FIG. 7A shows distribution of Bt spores displaying the GFP protein after BPER treatment (see Example 1).

FIG. 7B shows the fluorescence of Bt spores displaying the GFP protein, detected by flow cytometry (see Example 2).

FIG. 8A shows detection of spore-displayed CotB/TTFC antigen by flow cytometry and western blot.

FIG. 8B shows antibody titers following CotB/TTFC spore immunization.

FIG. 9 is an alignment of the amino acid sequences of Bt spore coat proteins CotB1 and CotB2 (SEQ ID NOS: 6 and 7, respectively).

FIG. 10 shows an alignment of the CotB1 and CotB2 amino acid sequences with the amino acid sequences of CotB proteins from other Bacilli, including other Bt strains (SEQ ID NOs: 13-21).

FIG. 11 shows the display of tetanus toxin fragment C (TTFC) on Bt spores (see Example 13).

FIG. 12 illustrates Bt spore coat protein constructs using a monomeric (A) and a dimeric (B) linker, respectively.

FIG. 13 illustrates the use of spores as an encoded support for conjugates or collections of molecules.

FIG. 14 illustrates positive cell selection with magnetic sorting (MACS)

FIG. 15 illustrates a method for simultaneous selection of conformational epitope and antibody libraries, where the conformational epitope library is presented using spore display while the antibody library is a phagemid library.

FIG. 16 shows that CotB-TTFC antigen spores specifically bind human anti-TTFC Phage.

FIG. 17 shows additional CotB sequences and sequence alignments (SEQ ID NOs: 22-45).

FIG. 18 shows the results of the survival of mice after challenge with tetanus toxin fragment C (TTFC).

FIG. 19 shows that IgG₂ response correlates with 72 hour survival.

FIG. 20 shows the coding sequence of human thrombopoietin (Tpo) as used in Example 17 (SEQ ID NO: 55).

FIG. 21 shows eight unique N-terminal peptides from 16 select Bt toxins (SEQ ID Nos: 46-53).

FIG. 22 shows amyloid A beta peptide (1-15) expressed on spores fused to cotB.

FIG. 23 shows Tpo recombinantly fused to the amino terminus of cotB and expressed on spores.

### DETAILED DESCRIPTION OF THE INVENTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

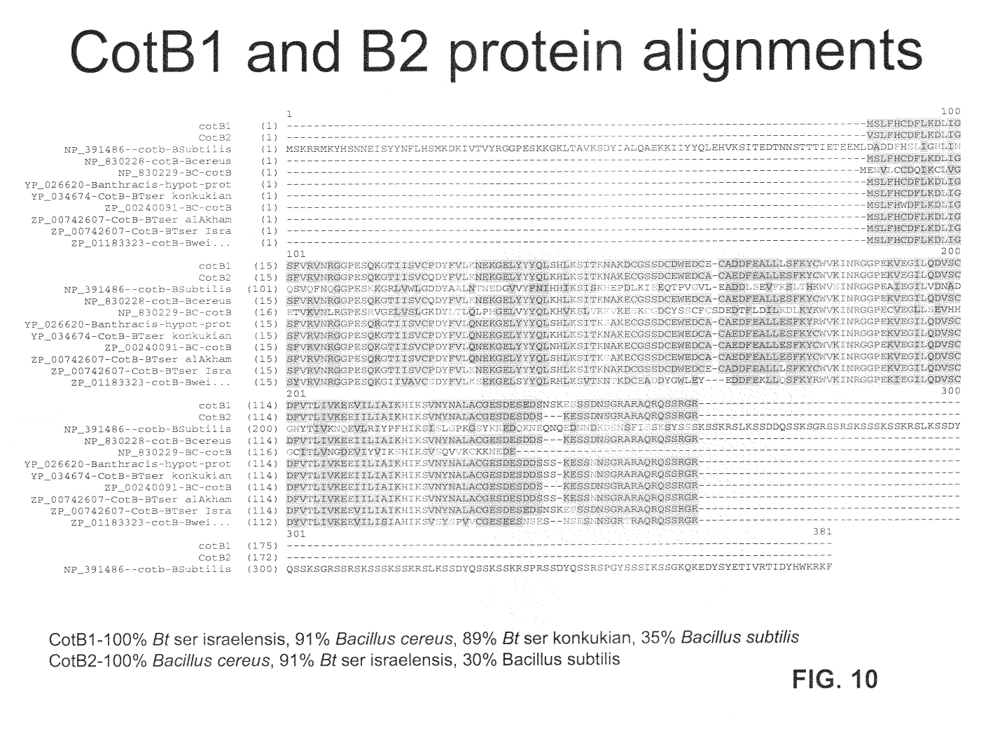

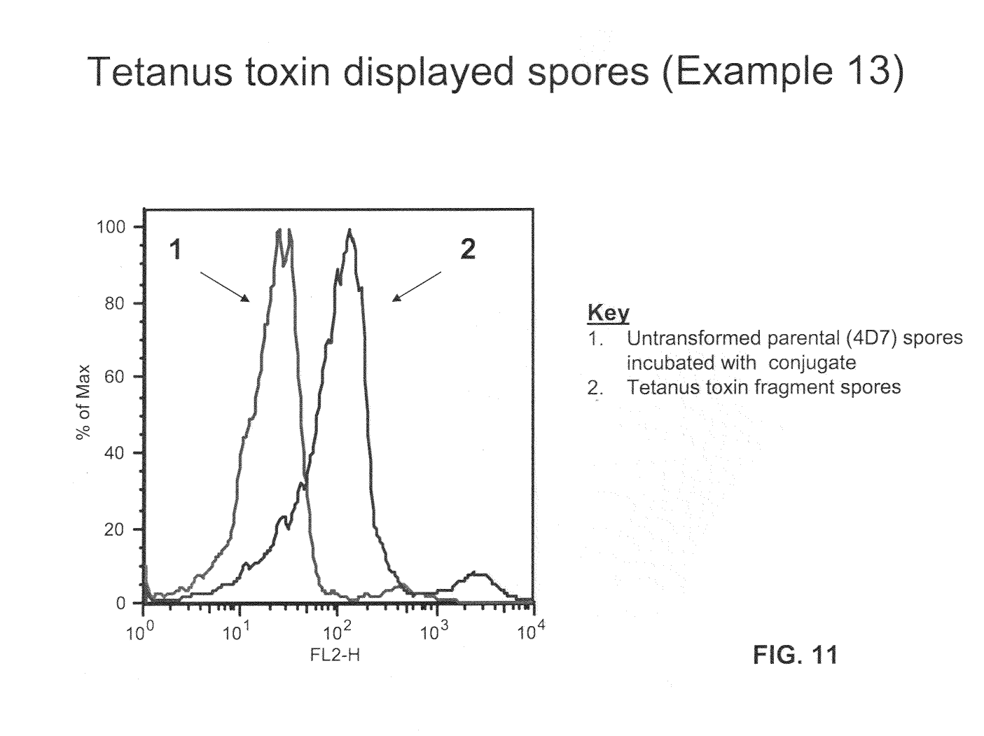

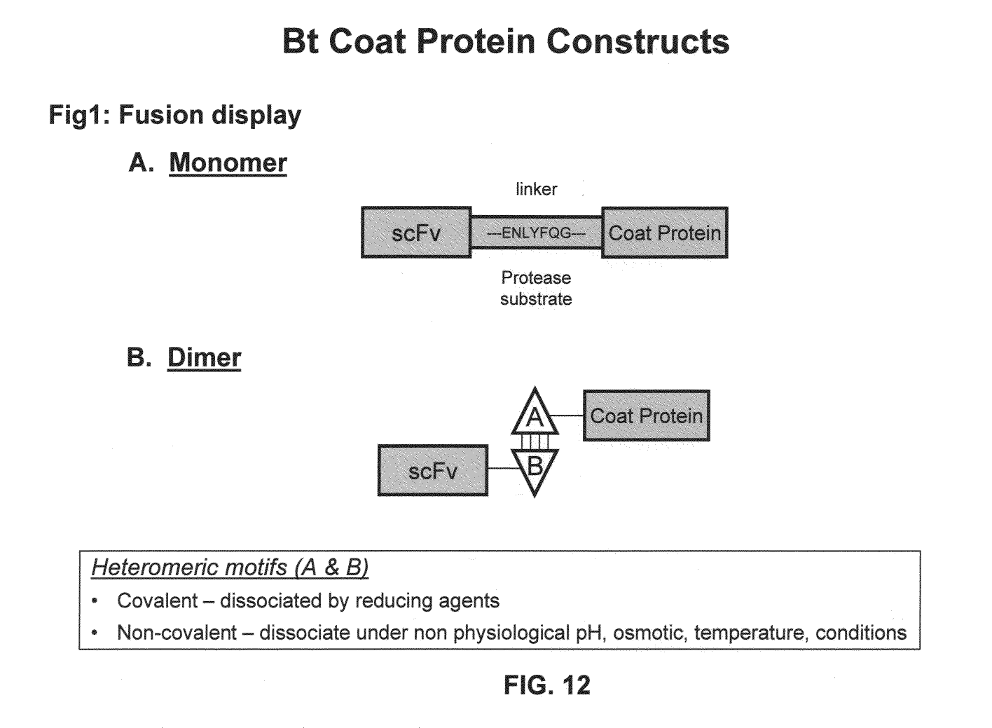

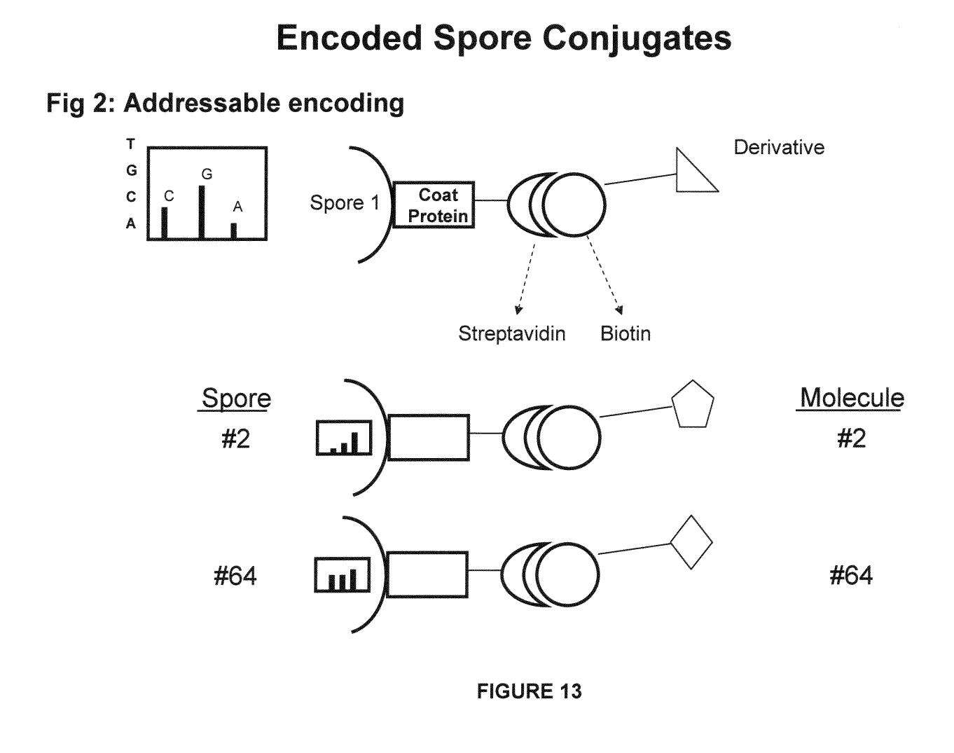

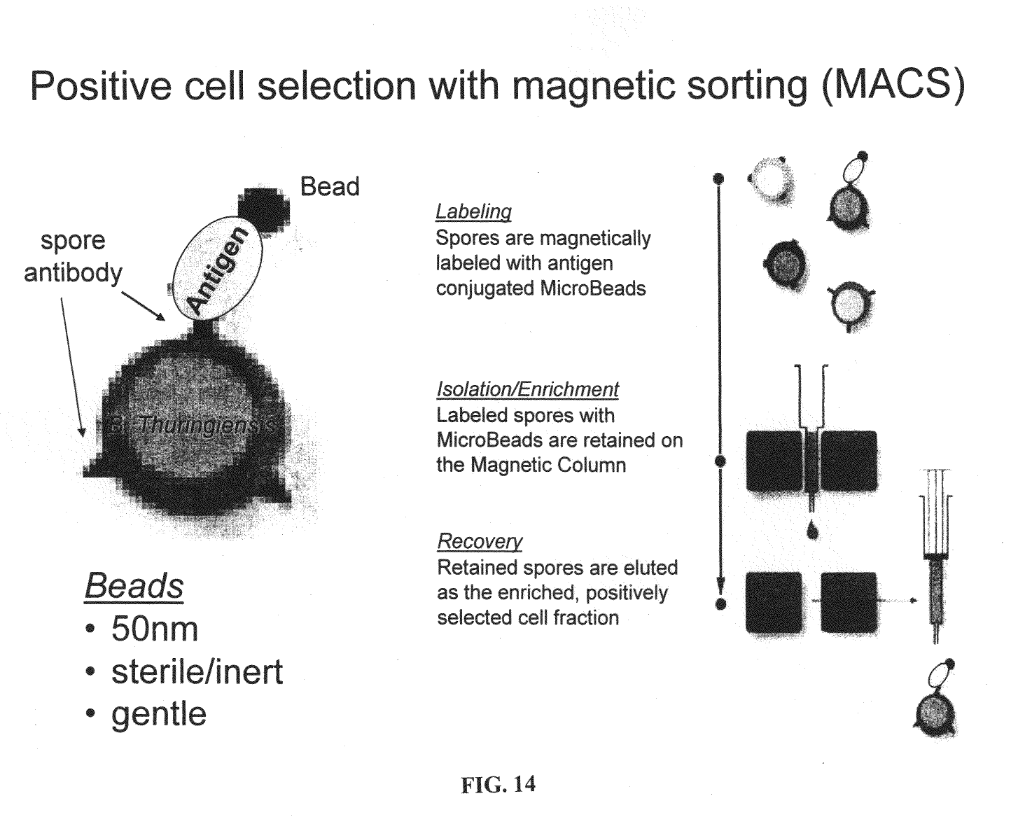

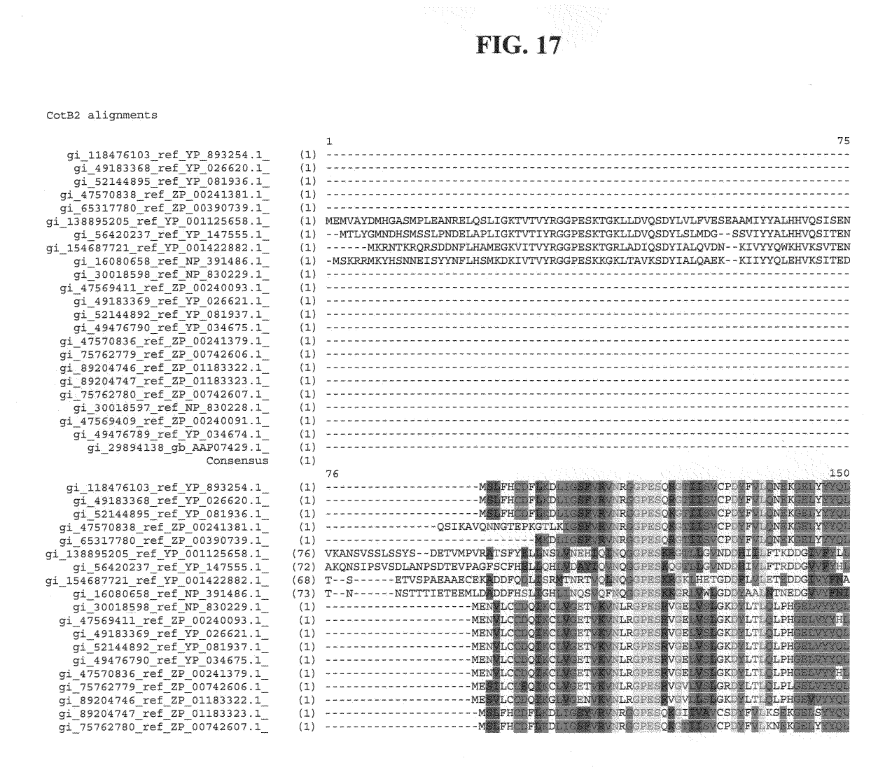

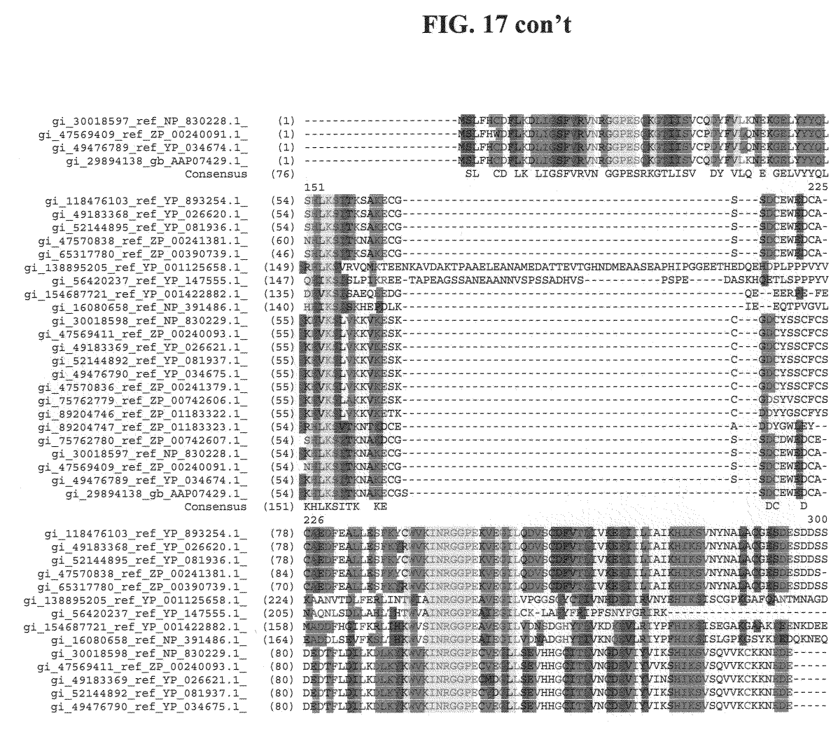

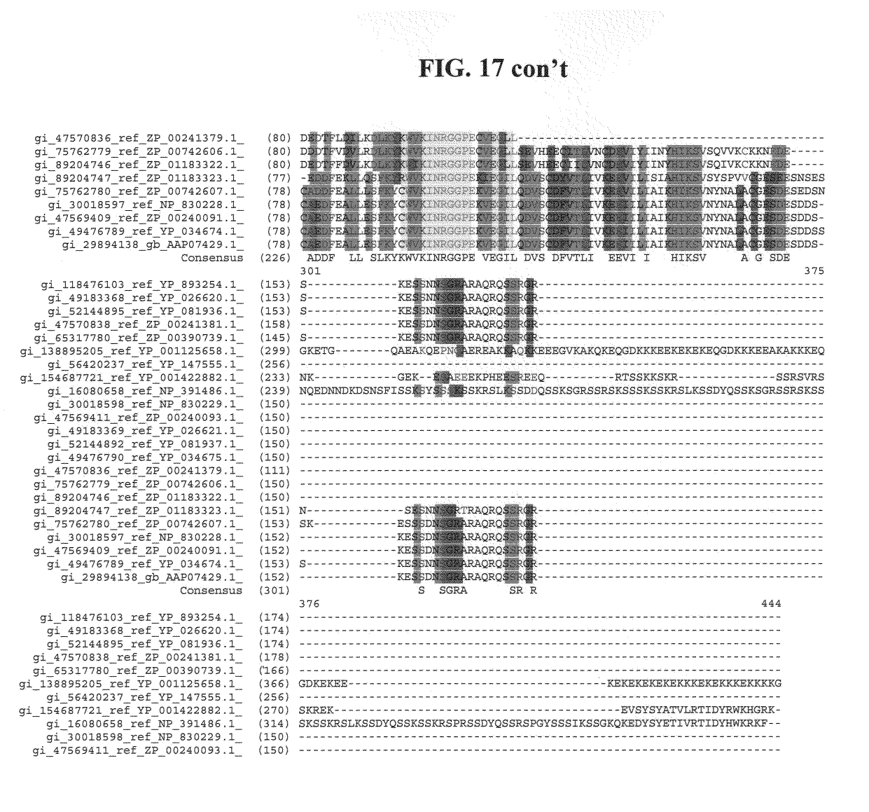

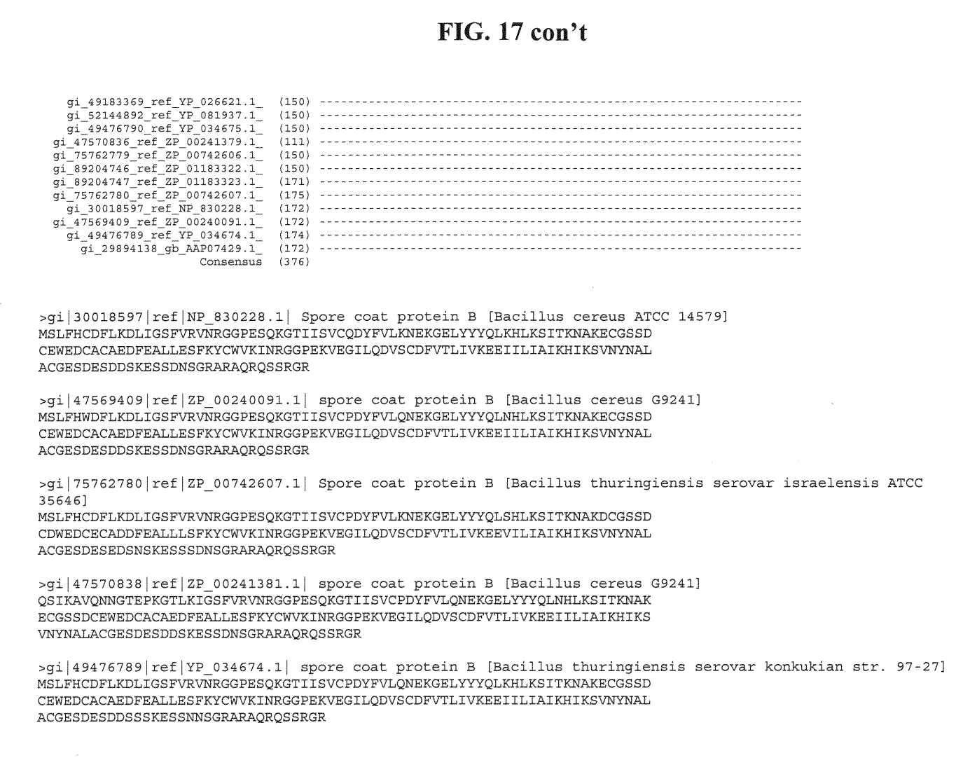

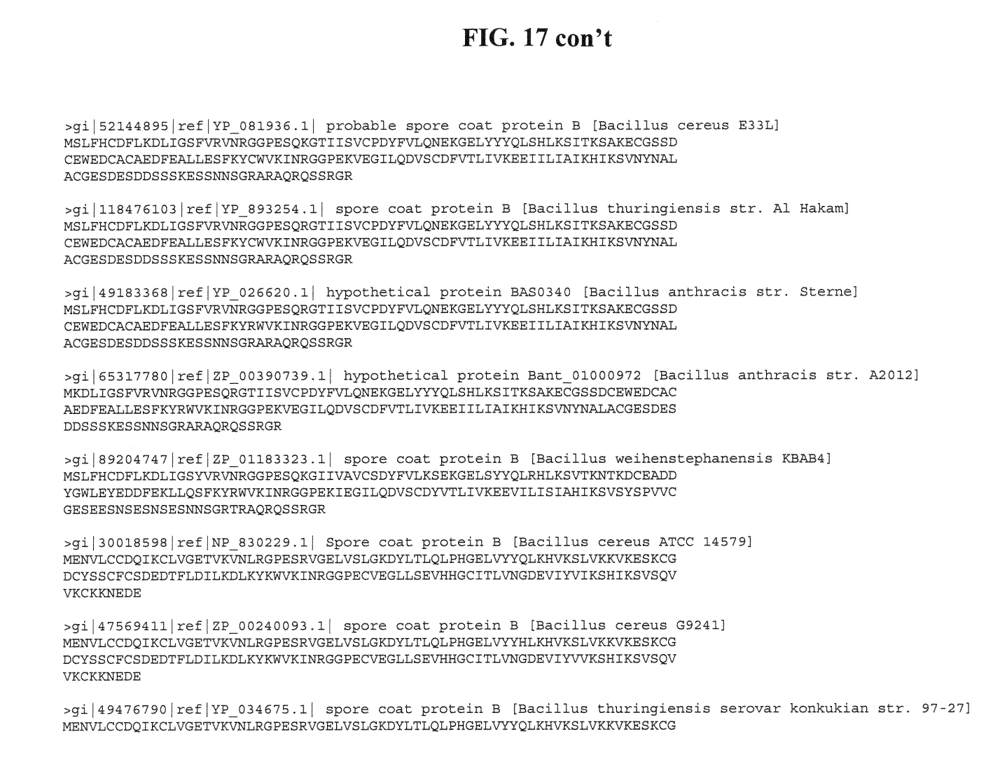

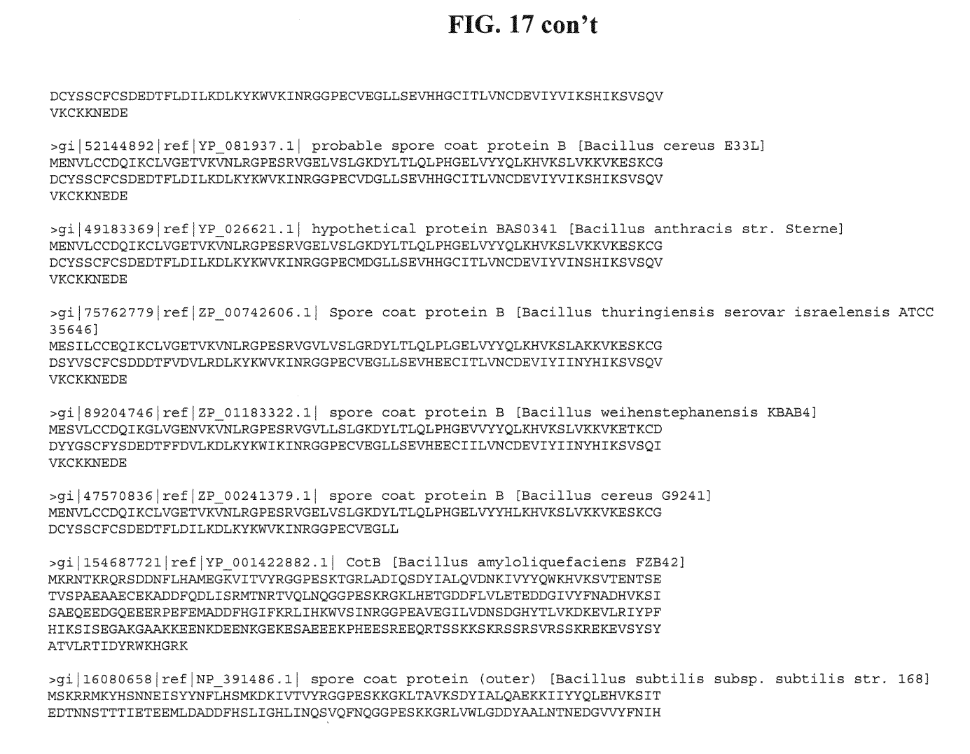

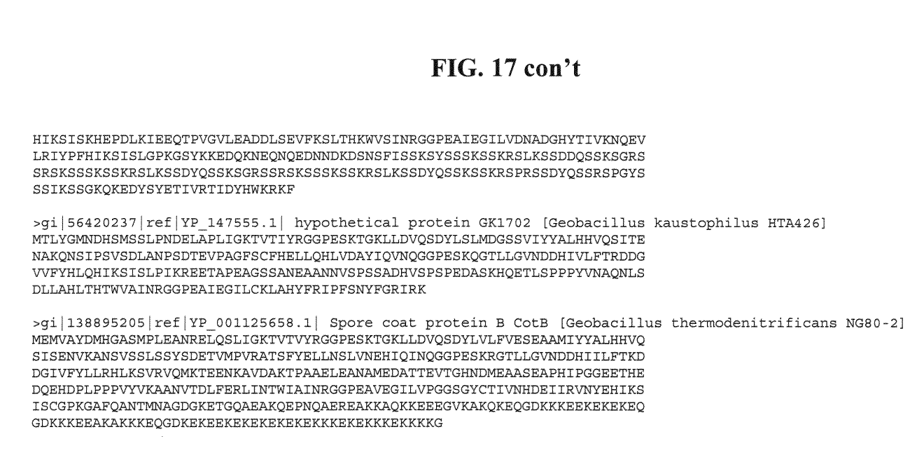

The term “externally exposed spore coat protein” is used herein in the broadest sense and includes any native protein present in the outer layer of spore coat and exposed on the spore surface, and functional fragments and functional amino acid sequence variants of such native proteins. The term includes native coat protein sequences of any spore-forming species and subspecies of the genus Bacillus, and functional fragments and functional amino acid sequence variants of such native coat protein sequences. The term “native” in this context is used to refer to native-sequence polypeptides, and does not refer to their origin or mode of preparation. Thus, native externally exposed spore coat proteins may be isolated from their native source but can also be prepared by other, e.g. synthetic and/or recombinant methods. Functional amino acid sequence variants specifically include chimeric variants, comprising fusions of two or more native externally exposed spore coat protein sequences, or fragments thereof, such as two or more sequences selected from the sequences shown in FIGS. 10 and 17, or fragments thereof. Preferred chimeric variants include the full-length CotB1 (SEQ ID NO: 6) and/or CotB2 (SEQ ID NO: 7) sequences of Bt, or fragments thereof.

The term “exposed on the spore surface” is used herein in the broadest sense and includes complete and partial exposure of a protein, such as a spore coat protein. Thus, proteins at least a portion of which is present in the outer layer of the spore coat are specifically included within this term.

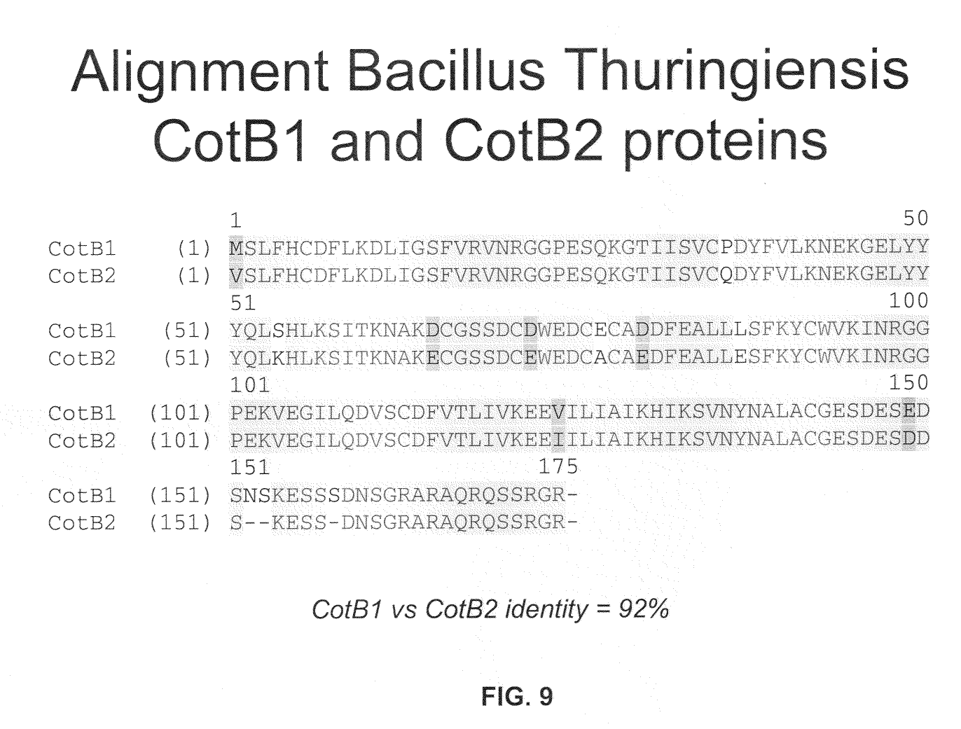

The terms “spore coat protein B” and “CotB protein” are used interchangeably, and refer to externally exposed spore coat proteins that are characterized by a highly hydrophobic region at the C-terminus, and classified as CotB, such as CotB1 or CotB2 proteins based on sequence homologies. Preferably, the CotB proteins herein show significant amino acid sequence identity to each other and to the amino terminal two-third of the 42.9-kDa component of the B. subtilis spore coat associated with the outer coat layer (Zheng et al., Genes Dev 2:1047-1057 (1988)). Sequences of representative CotB proteins herein are shown in FIGS. 9, 10, and 17 (SEQ ID Nos: 6, 7, and 13-45), which sequences are specifically included within the definition of spore coat protein B (CotB) herein. In this context, “significant sequence homology” means at least about 35%, or at least about 40%, or at least about 50%, preferably at least about 35% sequence identity to the amino terminal two-third of the 42.9-kDa component of the B. subtilis spore coat associated with the outer coat layer (Zheng et al., supra).

The term “Bacillus thuringiensis coat protein” or “Bt coat protein” is used herein to include externally exposed spore coat protein sequences of any subspecies of Bacillus thuringiensis, and functional fragments and functional amino acid sequence variants of such native sequences. Just as before, the term “native” in this context is used to refer to native-sequence polypeptides, and does not refer to their origin or mode of preparation. Thus, native Bt coat proteins may be isolated from their native source but can also be prepared by other, e.g. synthetic and/or recombinant methods. The term “native Bt coat protein” specifically includes, without limitation, CotB1 of SEQ ID NO: 6, CotB2 of SEQ ID NO: 7, and the other Bt CotB proteins shown in FIGS. 10 and 17 (SEQ ID Nos: 13-45), as well as functional fragments and functional chimeric variants thereof, comprising sequences from more than one native coat protein. Thus, the chimeric variants include, without limitation, chimeras comprising fusions of full-length CotB1 (SEQ ID NO: 6) and CotB2 (SEQ ID NO: 7) sequences or various fragments thereof, and chimeras comprising full-length CotB1 (SEQ ID NO: 6) and/or CotB2 (SEQ ID NO: 7) sequences, or fragments thereof, and chimeras comprising fusions of full-length cotB proteins of SEQ ID Nos: 13-45, or fragments thereof, in combination with sequences from other native coat proteins, including coat proteins from other Bt strains and/or from other Bacilli, such as Bacillus sublilis. The term “Bt coat protein” specifically includes, without limitation, functional amino acid sequence variants of the CotB1 (SEQ ID NO: 6) and CotB2 (SEQ ID NO: 7) Bt coat proteins, as well as functional amino acid sequence variants of other Bt CotB proteins, such as those shown in FIG. 10 or FIG. 17 (SEQ ID Nos: 13-45).

The terms “variant” and “amino acid sequence variant” are used interchangeably, and include substitution, deletion and/or insertion variants of native sequences. Specifically included within this definition are N- and/or C-terminal truncations of native CotB, protein sequences. In a preferred embodiment, the CotB protein variants have at least about 80% amino acid sequence identity, or at least about 85% amino acid sequence identity, or at least about 90% amino acid sequence identity, or at least about 92% amino acid sequence identity, or at least about 95% amino acid sequence identity, or at least about 95% amino acid sequence identity, or at least about 98% amino acid sequence identity with a native CotB sequence. Thus, in a preferred embodiment a variant of a native Bt coat protein has at least about 85% amino acid sequence identity, or at least about 90% amino acid sequence identity, or at least about 92% amino acid sequence identity, or at least about 95% amino acid sequence identity, or at least about 95% amino acid sequence identity, or at least about 98% amino acid sequence identity with the sequence of CotB1 (SEQ ID NO: 6) or CotB2 (SEQ ID NO: 7), or a functional fragment thereof.

A “functional” fragment or variant retains the ability to be propagated and stably displayed on the surface of a bacillus spore, such as a Bt spore.

The term “conjugate” or “conjugated” refers to any and all forms of covalent or non-covalent linkage, and includes, without limitation, direct genetic or chemical fusion, coupling through a linker or a cross-linking agent, and non-covalent associate, for example using a leucine zipper.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

As used herein, the terms “peptide,” “polypeptide” and “protein” all refer to a primary sequence of amino acids that are joined by covalent “peptide linkages.” In general, a peptide consists of a few amino acids, typically from about 2 to about 50 amino acids, and is shorter than a protein. The term “polypeptide” may encompass either peptides or proteins.

In the context of the present invention, the term “antibody” (Ab) is used in the broadest sense and includes polypeptides which exhibit binding specificity to a specific antigen as well as immunoglobulins and other antibody-like molecules which lack antigen specificity. Polypeptides of the latter kind are, for example, produced at low levels by the lymph system and, at increased levels, by myelomas. In the present application, the term “antibody” specifically covers, without limitation, monoclonal antibodies, polyclonal antibodies, and antibody fragments.

“Native antibodies” are usually heterotetrameric glycoproteins of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has, at one end, a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains, Chothia et al., J. Mol. Biol. 186:651 (1985); Novotny and Haber, Proc. Natl. Acad. Sci. U.S.A. 82:4592 (1985).

The term “variable” with reference to antibody chains is used to refer to portions of the antibody chains which differ extensively in sequence among antibodies and participate in the binding and specificity of each particular antibody for its particular antigen. Such variability is concentrated in three segments called hypervariable regions both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from a “complementarity determining region” or “CDR” (i.e., residues 30-36 (L1), 46-55 (L2) and 86-96 (L3) in the light chain variable domain and 30-35 (H1), 47-58 (H2) and 93-101 (H3) in the heavy chain variable domain; MacCallum et al., J Mol Biol. 1996.

The term “framework region” refers to the art recognized portions of an antibody variable region that exist between the more divergent CDR regions. Such framework regions are typically referred to as frameworks 1 through 4 (FR1, FR2, FR3, and FR4) and provide a scaffold for holding, in three-dimensional space, the three CDRs found in a heavy or light chain antibody variable region, such that the CDRs can form an antigen-binding surface.

Depending on the amino acid sequence of the constant domain of their heavy chains, antibodies can be assigned to different classes. There are five major classes of antibodies IgA, IgD, IgE, IgG, and IgM, and several of these may be further divided into subclasses (isotypes), e.g., IgG1, IgG2, IgG3, IgG4, IgA, and IgA2.

The heavy-chain constant domains that correspond to the different classes of immunoglobulins are called α, δ, ε, γ, and μ, respectively.

The “light chains” of antibodies from any vertebrate species can be assigned to one of two clearly distinct types, called kappa (κ) and lambda (λ), based on the amino acid sequences of their constant domains.

“Antibody fragments” comprise a portion of a full length antibody, generally the antigen binding domain(s) or variable domain(s) thereof. Examples of antibody fragments include, but are not limited to, Fab, Fab′, F(ab′)₂, scFv, (scFv)₂, dAb, and complementarity determining region (CDR) fragments, linear antibodies, single-chain antibody molecules, minibodies, diabodies, multispecific antibodies formed from antibody fragments, and, in general, polypeptides that contain at least a portion of an immunoglobulin that is sufficient to confer specific antigen binding to the polypeptide. Specifically within the scope of the invention are bispecific antibody fragments.

The terms “bispecific antibody” and “bispecific antibody fragment” are used herein to refer to antibodies or antibody fragments with binding specificity for at least two targets. If desired, multi-specificity can be combined by multi-valency in order to produce multivalent bispecific antibodies that possess more than one binding site for each of their targets. For example, by dimerizing two scFv fusions via the helix-turn-helix motif, (scFv)₁-hinge-helix-turn-helix-(scFv)₂, a tetravalent bispecific miniantibody was produced (Müller et al., FEBS Lett. 432(1-2):45-9 (1998)). The so-called “di-bi-miniantibody” possesses two binding sites to each of it target antigens.

The term “monoclonal antibody” is used to refer to an antibody molecule synthesized by a single clone of B cells. The modifier “monoclonal” indicates the character of the antibody as being obtained from a substantially homogeneous population of antibodies, and is not to be construed as requiring production of the antibody by any particular method. Thus, monoclonal antibodies may be made by the hybridoma method first described by Kohler and Milstein, Nature 256:495 (1975); Eur. J. Immunol. 6:511 (1976), by recombinant DNA techniques, or may also be isolated from phage antibody libraries.

The term “polyclonal antibody” is used to refer to a population of antibody molecules synthesized by a population of B cells.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Plückthun in The Pharmacology of Monoclonal Antibodies, vol. 113, Rosenburg and Moore eds. Springer-Verlag, New York, pp. 269-315 (1994). Single-chain antibodies are disclosed, for example in WO 88/06630 and WO 92/01047.

As used herein the term “antibody binding regions” refers to one or more portions of an immunoglobulin or antibody variable region capable of binding an antigen(s). Typically, the antibody binding region is, for example, an antibody light chain (VL) (or variable region thereof), an antibody heavy chain (VH) (or variable region thereof), a heavy chain Fc region, a combined antibody light and heavy chain (or variable region thereof) such as a Fab, F(ab′)₂, single domain, or single chain antibody (scFv), or a full length antibody, for example, an IgG (e.g., an IgG1, IgG2, IgG3, or IgG4 subtype), IgA1, IgA2, IgD, IgE, or IgM antibody.

The term “amino acid” or “amino acid residue” typically refers to an amino acid having its art recognized definition such as an amino acid selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val) although modified, synthetic, or rare amino acids may be used as desired. Thus, modified and unusual amino acids listed in 37 CFR 1.822(b)(4) are specifically included within this definition and expressly incorporated herein by reference. Amino acids can be subdivided into various sub-groups. Thus, amino acids can be grouped as having a nonpolar side chain (e.g., Ala, Cys, Ile, Leu, Met, Phe, Pro, Val); a negatively charged side chain (e.g., Asp, Glu); a positively charged side chain (e.g., Arg, His, Lys); or an uncharged polar side chain (e.g., Asn, Cys, Gln, Gly, His, Met, Phe, Ser, Thr, Trp, and Tyr). Amino acids can also be grouped as small amino acids (Gly, Ala), nucleophilic amino acids (Ser, His, Thr, Cys), hydrophobic amino acids (Val, Leu, Ile, Met, Pro), aromatic amino acids (Phe, Tyr, Trp, Asp, Glu), amides (Asp, Glu), and basic amino acids (Lys, Arg) (see, FIG. 25).

The term “polynucleotide(s)” refers to nucleic acids such as DNA molecules and RNA molecules and analogs thereof (e.g., DNA or RNA generated using nucleotide analogs or using nucleic acid chemistry). As desired, the polynucleotides may be made synthetically, e.g., using art-recognized nucleic acid chemistry or enzymatically using, e.g., a polymerase, and, if desired, be modified. Typical modifications include methylation, biotinylation, and other art-known modifications. In addition, the nucleic acid molecule can be single-stranded or double-stranded and, where desired, linked to a detectable moiety.

The term “mutagenesis” refers to, unless otherwise specified, any art recognized technique for altering a polynucleotide or polypeptide sequence. Preferred types of mutagenesis include error prone PCR mutagenesis, saturation mutagenesis, or other site directed mutagenesis.

The term “vector” is used to refer to a rDNA molecule capable of autonomous replication in a cell and to which a DNA segment, e.g., gene or polynucleotide, can be operatively linked so as to bring about replication of the attached segment. Vectors capable of directing the expression of genes encoding for one or more polypeptides are referred to herein as “expression vectors.”

Percent amino acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

The term “leucine zipper” is used to refer to a repetitive heptad motif typically containing four to five leucine residues present as a conserved domain in several proteins. Leucine zippers fold as short, parallel coiled coils, and are believed to be responsible for oligomerization of the proteins of which they form a domain.

The term “epitope” as used herein, refers to a sequence of at least about 3 to 5, preferably at least about 5 to 10, or at least about 5 to 15 amino acids, and typically not more than about 500, or about 1,000 amino acids, which define a sequence that by itself, or as part of a larger sequence, binds to an antibody generated in response to such sequence. An epitope is not limited to a polypeptide having a sequence identical to the portion of the parent protein from which it is derived. Indeed, viral genomes are in a state of constant change and exhibit relatively high degrees of variability between isolates. Thus the term “epitope” encompasses sequences identical to the native sequence, as well as modifications, such as deletions, substitutions and/or insertions to the native sequence. Generally, such modifications are conservative in nature but non-conservative modifications are also contemplated. The term specifically includes “mimotopes,” i.e. sequences that do not identify a continuous linear native sequence or do not necessarily occur in a native protein, but functionally mimic an epitope on a native protein. The term “epitope” specifically includes linear and conformational epitopes.

As used herein, the term “conformational epitope” refers to an epitope formed by discontinuous portions of a protein having structural features of corresponding sequences in the properly folded full-length native protein. The length of the epitope-defining sequence (the sequence including the discontinuous portions making up the conformational epitope) can greatly vary as these epitopes are formed by the three-dimensional structure of the protein. Thus, amino acids defining the epitope can be relatively few in number, widely dispersed along the length of the molecule, being brought into correct epitope conformation via folding. The portions of the protein between the residues defining the epitope may not be critical to the conformational structure of the epitope. For example, deletion or substitution of these intervening sequences may not affect the conformational epitope provided that the sequences critical to epitope conformation are maintained. Thus, a “conformational epitope,” as defined herein, is not required to be identical to a native conformational epitope, but rather includes conformationally constrained structures that regenerate (exhibit) essential properties (such as qualitative antibody-binding properties) of native conformational epitopes.

“Linear epitopes” are fragments of discontinuous or conformational epitopes.

The terms, “SURROBODY™”, “surrobody,” and “surrogate light chain construct” are used in the broadest sense and include antibody surrogate light chain-based (pre-BCR-based) polypeptide structures, which are typically capable of binding to target sequences (generally referred to as “antigens” in the context of antibodies), as disclosed in Xu et al., Proc Natl Acad Sci USA, 105(31):10756-61 (2008), and in co-pending PCT Application No. PCT/US2008/058283 filed Mar. 26, 2008, the entire disclosures of which are hereby expressly incorporated by reference.

The term “surrogate light chain,” as used herein, refers to a dimer formed by the non-covalent association of a VpreB and a λ5 protein.

For the three-dimensional structure of the pre-B-cell receptor (pre-BCR), including the structure of the surrogate light chain (SCL) and its components see, e.g. Lanig et al., Mol. Immunol. 40(17):1263-72 (2004).

### B. Detailed Description

Techniques for performing the methods of the present invention are well known in the art and described in standard laboratory textbooks, including, for example, Molecular Biological Methods for Bacillus, Hardwood and Cutting, eds., John Wiley & Sons, 1990; Ausubel et al., Current Protocols of Molecular Biology, John Wiley and Sons (1997); Molecular Cloning: A Laboratory Manual, Third Edition, J. Sambrook and D. W. Russell, eds., Cold Spring Harbor, N.Y., USA, Cold Spring Harbor Laboratory Press, 2001; O'Brian et al., Analytical Chemistry of Bacillus Thuringiensis, Hickle and Fitch, eds., Am. Chem. Soc., 1990; Bacillus thuringiensis: biology, ecology and safety, T. R. Glare and M. O'Callaghan, eds., John Wiley, 2000; Antibody Phage Display, Methods and Protocols, Humana Press, 2001; and Antibodies, G. Subramanian, ed., Kluwer Academic, 2004. Mutagenesis can, for example, be performed using site-directed mutagenesis (Kunkel et al., Proc. Natl. Acad. Sci. USA 82:488-492 (1985)). PCR amplification methods are described in U.S. Pat. Nos. 4,683,192, 4,683,202, 4,800,159, and 4,965,188, and in several textbooks including “PCR Technology: Principles and Applications for DNA Amplification”, H. Erlich, ed., Stockton Press, New York (1989); and PCR Protocols: A Guide to Methods and Applications, Innis et al., eds., Academic Press, San Diego, Calif. (1990).

The present invention concerns a spore display system for surface display of heterologous peptides or polypeptides on bacillus spores. The spore display system of the present invention utilizes externally exposed spore coat proteins or fragments thereof as fusion partners for display of heterologous molecules, in particular heterologous peptides and polypeptides, on the surface of spores.

The coat of bacterial spores is a biochemically complex structure, which is composed of about 40-60 proteins in B. subtilis, B. anthracis, and probably most other species (Kuwana et al., Microbiology 148:3971-3982 (2002); Lai et al., J. Bacteriol. 185:1443-1454 (2003)). The bacterial coat proteins have a variety of biological functions. Thus, for example CotA, CotB and CotE have been attributed a role in spore morphology, and CotA and CotB have also been described to play a role in ridge pattern formation (Chada et al., J. Bacteriol. 185(210:6255-6261 (2003)). Spore coat proteins, similar to those found on Bacillus cereus spores, were identified from Bacillus thuringiensis kurstaki as early as 1982 (Aronson et al., J. Bacteriol. 151(1):399-410 (1982)).

Externally displayed coat proteins of any genus of Bacilli are suitable for use in the methods of the present invention. Representative Bacilli species include, without limitation, Bacillus anthracis, such as Bacillus anthracis strain Sterne or strain A2012; Bacillus amyloliquefaciens, such as strain FZB42; Bacillus cereus ATCC 14579; Bacillus cereus G9241; Bacillus cereus E33L; Bacillus subtilis, such as subsp. subtilis strain 168; Bacillus thuringiensis, such as Bacillus thuringiensi serovar israelensis ATCC 35646, Bacillus thuringiensis serovar konkukian str. 97-27; Bacillus thuringiensis str. Al Hakam; Bacillus weihenstephanensis KBAB4; Geobacillus kaustophilus HTA426; and Geobacillus thermodenitrificans NG80-2.

Sequences of certain CotB proteins are shown in FIGS. 9, 10 and 17.

In a particular embodiment, the externally exposed CotB protein is a Bt coat protein, such as CotB1 of Bt (SEQ ID NO: 6, encoded by the nucleic acid of SEQ ID NO: 1), or a functional fragment or variant thereof, or CotB2 of Bt (SEQ ID NO: 7, encoded by the nucleic acid of SEQ ID NO: 2), or a functional fragment or variant thereof. It will be understood, however, that different Bt subspecies may contain coat proteins with different sequences, and the use of such different coat proteins, as well as their functional fragments and variants, is also within the scope of the present invention. Coat proteins from other Bt subspecies, such as, konkukian, alakham, israelensis, are shown in FIGS. 10 and 17. In addition, the Bt coat protein sequences of the present invention include chimeric molecules comprising fusions of various Bt coat proteins or fragments thereof, or fusions of Bt coat proteins or fragments thereof with parts or whole of coat proteins from other bacilli, such as Bacillus subtilis, or any other Bacilli listed in FIGS. 10 and 17.

In a particular embodiment, the externally exposed coat protein of the present invention is other than a CotB protein of Bacillus subtilis, and specifically other than the CotB protein described by Isticato et al., J. Bacteriol. 183:6294-6301 (2001).

In another embodiment, the externally exposed coat protein of the present invention is other than a fragment of a CotB protein of Bacillus subtilis, and specifically other than a CotB protein fragment described by Isticato et al., J. Bacteriol. 183:6294-6301 (2001).

The coat protein sequences of the present invention can be obtained from their native sources, i.e. spore coats of various Bacilli, including various species and subspecies, produced by chemical synthesis or methods of recombinant DNA technology, or by any other technique known in the art, or by a combination of two or more of such techniques. Native coat proteins or their coring sequences can be isolated from various species and subspecies, including those listed above or shown in FIGS. 10 and 17. Thus, for example, native Bt coat proteins or their coding sequences can be isolated from various Bt subspecies, such as, subspecies kurstaki, dendrolimus, galleriae, entomocidus, aizawai, morrisoni, tolworthi, alesti, or israelensis.

Thus, DNA encoding the coat proteins or fragments thereof can be PCR amplified from the chromosome of a suitable bacillus using appropriate oligonucleotide primers and probe, by methods known in the art. The PCR product can then be purified by known techniques, such as, for example, by using the QIAquick gel extraction kit (Qiagen) following the manufacturer's instructions.

Recombinant host cells suitable for cloning the coat protein fragments herein include prokaryote, yeast, or higher eukaryote cells. For cloning and routine plasmid manipulation the preferred host is E. coli.

The coat protein sequences of the present invention can be used to display heterologous molecules on the surface of spores of spore-forming bacilli. Thus, the present invention also concerns conjugates of coat protein sequences to molecules to be displayed. Examples of molecules that can be displayed using the coat protein sequences herein include, without limitation, peptides and polypeptides, such as, for example, receptors, ligands, antibodies and antibody fragments, surrobodies and surrobody fragments, and enzymes, peptides and vaccines. Essentially all polypeptides and peptides can be displayed on the surface of spores following the methods of the present invention. Similarly, the spore display technique of the present invention allows display of non-peptide small organic molecules as well.

Without limitation, the antibodies and antibody fragments, or surrobodies or surrobody fragments, displayed in accordance with the present invention may bind to polypeptides including cell surface and soluble receptors, cytokines, growth factors, enzymes; proteases; and hormones, for example. Thus, the antibody or surrobody may bind to a cytokine, such as tumor necrosis factor-α, or -β (TNF-α and -β), an interleukin, e.g. IL-1, IL-2, IL-3, IL-4, IL-5, IL-6, IL-10, IL-11, IL-12, IL-15, IL-17, IL-18, IL-23, and their respective family members, interferons-α, -β, and -γ (IFN-α, -β, and -γ), including their respective sub-species, TWEAK, RANKL, BLys, RANTES, MCP-1, MIP-1α, MIP-1β, SDF-1, colony stimulating factor (CSF), granulocyte colony stimulating factor (G-CSF), or granulocyte macrophage colony stimulating factor (GMCSF). The antibodies displayed in accordance with the present invention may also bind a growth factor, including, without limitation, vascular endothelial growth factor (VEGF), nerve growth factor (NGF), insulin-like growth factor 1 (IGF-1), epidermal growth factor (EGF), plateled derived growth factor (PDGF), placental growth factor (PLGF), tissue growth factor-α (TGF-α), and tissue growth factor-β (TGF-β). Other exemplary antibodies include antibodies to CD proteins such as CD3, CD4, CD8, CD19, CD20, or CD22; erythropoietin (EPO); thrombopoietin (TPO), osteoinductive factors; immunotoxins; a bone morphogenetic protein (BMP); T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as EGFR (HER1/ErbB1), HER2/ErbB2, HER3/ErbB or HER4/ErbB4 receptor, other receptor polypeptides, such as TNFR1, TNFR2, EpoR1, GHBP, or hormones, such as growth hormone (GH). An example of enzymes that can be displayed herein is the E. coli beta-galactosidase enzyme.

The proteins that can be displayed in accordance with the present invention include multimeric proteins. Thus coat protein fusions of individual chains of proteins that form homo- and/or heterodimers can be co-expressed in a proximity that allows the formation of such homo- and/or heterodimers. There are many proteins known to form homo- and/or heterodimers, including, for example, G-protein-coupled receptors, members of the tyrosine kinase receptor, chemokine and cytokine families, enzyme complexes, and transcriptional factors. In addition, the function of most filamentous proteins of the cytoskeleton, such as actin, myosin, spectrin, tubulin, etc., relies on their oligomerization or polymerization. One specific example of proteins known to form homodimers is the erythropoietin (EPO) receptor. An example of heterodimeric proteins the expression of antibody heavy and light chains, such as, for example, the heavy and light chains of a Fab fragment, as coat protein fusion, which can then form a heterodimer with cognate antigen binding capabilities. While it is possible to anchor both (or all) components of a dimer (or multimer) to the spore, using the conjugates of the present invention, according to another embodiment, only one participant is anchored to the spore while the other participant is coexpressed without such anchoring, and allowed to assemble with the participant anchored to the spore through natural associations within the sporulating cell. In another embodiment, both (or all) components of a dimer (or multimer) are anchored to the spore, where the anchor spore coat protein sequences may be identical or different. For example, it is possible to fuse an antibody heavy chain or a fragment thereof to CotB1 of SEQ ID NO: 6 or a functional fragment or variant thereof, and fuse the light chain of the same antibody, or a fragment thereof, to CotB2 of SEQ ID NO: 7, or a functional fragment or variant thereof. Similar structures can be prepared with the coat protein sequences shown in FIG. 10 and/or FIG. 17.

The antibodies displayed may include therapeutic antibodies or antibodies with therapeutic and/or diagnostic potential, that may be commercially available or under development, such as, for example, D2E7 (adalimumab, Abbott), a fully human anti-TNF-α antibody for the treatment of rheumatoid arthritis (RA); an anti-VEGF antibody (e.g. A.461), or the anti-TPO antibody Tn1. Apart from complete antibodies, single-chain antibodies, antibody fragments, and antibody-like molecules can also be displayed in accordance with the present invention.

Similarly, the surrobodies and surrobody fragments displayed include therapeutic surrobodies, which may, for example, bind to any of the target polypeptides listed above, and may have therapeutic applications similar to therapeutic antibodies, such as those specifically listed above.

Peptides that can be displayed on Bt spores following the methods of the present invention include, for example, Myc, His6, and FLAG.

In addition, vaccines, such as a tetanus toxin fragment C (TTFC, see, e.g. FIG. 5, SEQ ID NO: 4), or B subunit of heat-labile enterotoxin of E. coli (LTB) can be included in the coat protein fusions herein and displayed in accordance with the present invention.

When the heterologous molecule is a peptide or a polypeptide, including antibodies, antibody fragments, enzymes and vaccines, conjugation may be by fusion, preferably at a terminal end, such as the N- and/or C-terminus of the coat protein, such as a Bt coat protein (e.g. CotB1 or CotB2) or any other coat protein sequence herein, specifically including, without limitation, the sequences shown in FIGS. 10 and 17. Alternatively, an appropriate peptide linker sequence can be used to prepare the conjugates.

The linker sequence separates the displayed molecule (e.g. polypeptide) and the coat protein sequence by a distance sufficient to ensure that each sequence properly folds into its secondary and tertiary structures. The length of the linker sequence may vary and generally is between 1 and about 50 amino acids, more commonly, up to about 15 amino acids, or up to about 10 amino acids, or up to about 8 amino acids, or up to about 7 amino acids, or up to about 5 amino acids, or up to about 3 amino acids long. The linker sequence is incorporated into the conjugate by methods well known in the art.

In order to facilitate removal of the displayed molecule, such as an scFv antibody fragment, the linker may include a sequence that is a substrate for an enzyme, such as a protease. Thus, in a specific embodiment, the natural substrate of a given protease can be used as or included in the linker peptide. For instance, the linker can be or can include the substrate site of the tobacco etch virus (TEV) (ENLYFOG) (SEQ ID NO: 8). Alternatively, the linker peptide may be different from the natural substrate of a protease, but may include sequences that can be cleaved by the protease. Thus, it is known that trypsin-like proteases specifically cleave at the carboxyl side of lysine and arginine residues, while chymotrypsin-like proteases are specific for cleavage at tyrosine, phenylalanine and tryptophan residues, etc.

The linkage between the coat protein sequence and the molecule to be displayed (such as a scFv), can be achieved by using a heterodimeric motif, where the two components forming the dimer, designated as “A” and “B” can be binding partners which are covalently associated with each other, or may associate through non-covalent interaction.

Covalent association may, for example, take place through the formation of a disulfide bond between cysteines of the binding partners. The disulfide bond can be broken and the displayed molecule (e.g. scFv) released by treatment with a reducing agent that disrupts the disulfide bond, such as, for example, dithiothreitol, dithioerythritol, β-mercaptoethanol, phosphines, sodium borohydride, and the like. Preferably, thiol-group containing reducing agents are used.

Non-covalent association can be achieved, for example, using a pair of leucine zipper peptides. Leucine zippers were originally identified in several DNA-binding proteins (Landschulz et al., Science 240: 1759, 1988). Thus, the leucine zipper domain is a term used to refer to a conserved peptide domain present in these proteins, which is responsible for dimerization of the proteins. The leucine zipper domain comprises a repetitive heptad repeat, typically with four or five leucine residues interspersed with other amino acids.

Leucine zipper peptides include, for example, the well known

c-Jun “leucine zipper peptide”

(SEQ ID NO: 9)

RIARLEEKVKTLKAQNSELASTANMLREQVAQLKQKVMNY
and the
v-Fos “leucine zipper peptide”

(SEQ ID NO: 10)

LTDTLQAETDQLEDKKSALQTEIANLLKEKEKLEFILAAY.

The products of the nuclear oncogenes fos and jun comprise leucine zipper domains which preferentially form a heterodimer (O'Shea et al., Science 245:646, 1989; Turner and Tjian, Science 243:1689, 1989).

Other examples of leucine zipper peptides include, without limitation, domains found in the yeast transcription factor GCN4 and a heat-stable DNA-binding protein found in rat liver (C/EBP; Landschulz et al., Science 243:1681, 1989); the gene product of the murine proto-oncogene, c-myc (Landschulz et al., Science 240:1759, 1988). The fusogenic proteins of several different viruses, including paramyxovirus, coronavirus, measles virus and many retroviruses, also possess leucine zipper domains (Buckland and Wild, Nature 338:547, 1989; Britton, Nature 353:394, 1991; Delwat and Mosialos, AIDS Research and Human Retroviruses 6:703, 1990). It is often preferred to use synthetic, as opposed to naturally occurring, leucine zipper peptides, since the synthetic sequences can be designed to exhibit improved properties, such as stability.

In order to produce the fusions of the present invention, the amplified coat protein-encoding DNA can be cloned into an appropriate plasmid in frame with the coding sequence of the peptide or polypeptide to be displayed, under control of a suitable sporulation-specific promoter. The sporulation specific promoter can, but does not have to be, obtained from the same species or subspecies from which the coat protein originates.

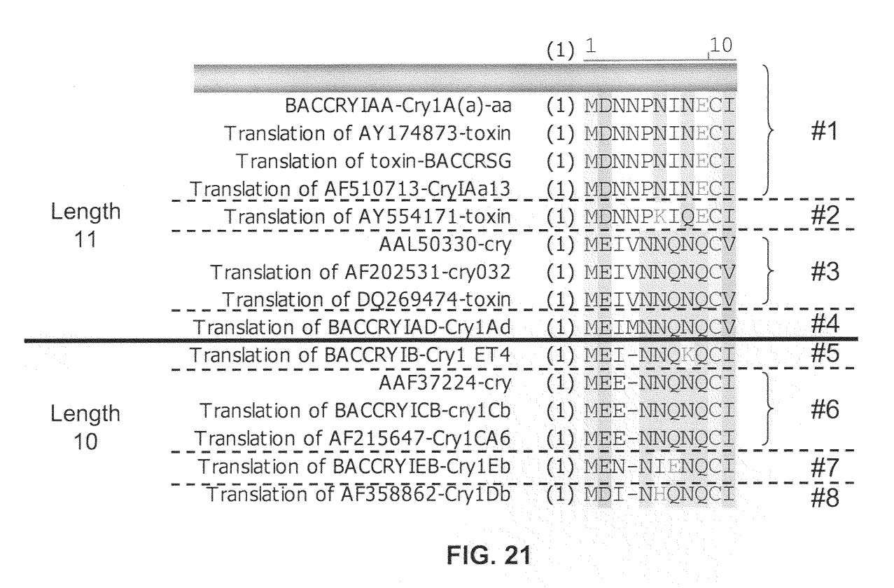

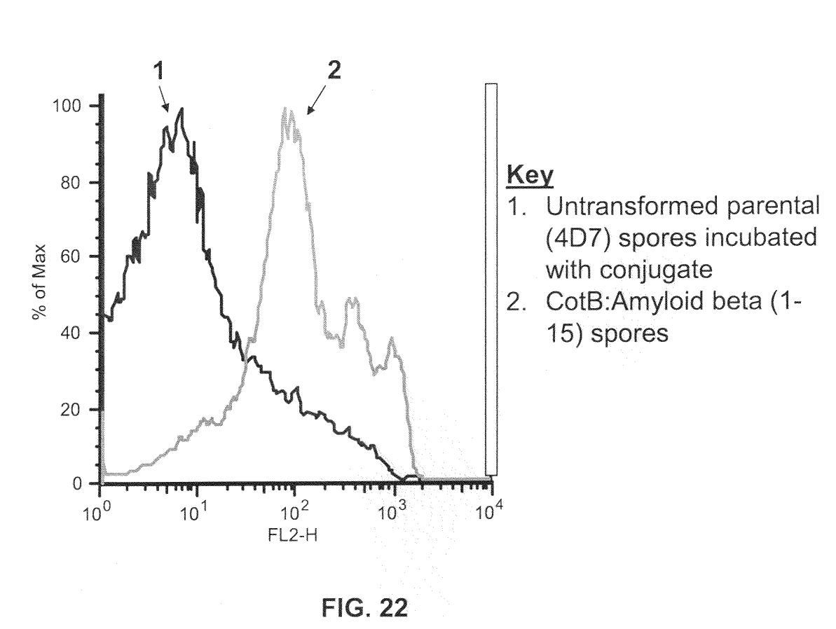

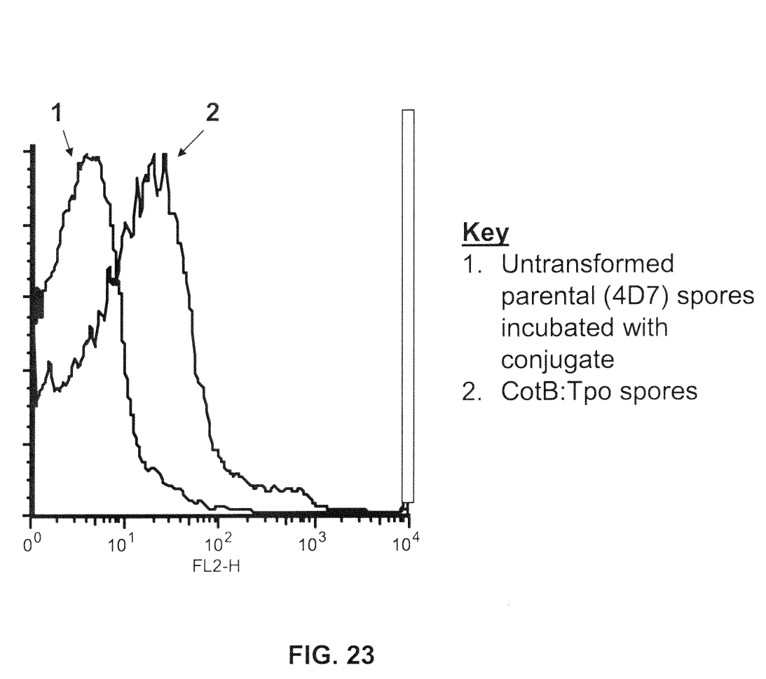

In a preferred embodiment, the coding sequence of the mature spore code protein is preceded by the coding sequence of a short N-terminal peptide, such as, for example, one of the peptides shown in FIG. 21. The length of the N-terminal peptide is typically between about 8 to about 20 amino acids, such as, for example, about 10-15, or about 10-12, or about 10 or 11 amino acids. The N-terminal peptide may originate from the pro-sequence of the coat protein included in the fusion, or may be from a different toxin. Artificial sequences not occurring in nature, such as for example, consensus sequences from native N-terminal peptides, are also contemplated for use in the fusions of the present invention.

The plasmids containing the coding sequences for the coat protein-heterologous peptide/polypeptide fusions can be introduced into Bacillus by electroporation, essentially as described by Du et al., Appl. Environ. Microbiol. 71(6):3337-3341 (2005), following the method of Macaluso and Mettus, J. Bacteriol. 173:1353-1356 (1991).

After plasmid transformation of the target strain, the cells are grown in an appropriate medium to promote sporulation. Traditionally, 48-72 hours are required to efficiently convert and liberate spores from a vegetative population of cells. However, periods lasting longer than about 24 hours result in appreciable reduction in coat-fusion protein stability. As after about 12-18 hours significant levels of coat-fusion protein are present on the endospore surface it has been found that it is beneficial to liberate these recombinant spores from vegetative mother cells. Such liberation can be accomplished by detergent lysis of the mother cells. As a result of the development of a spore liberation step, not only are spores harvested more quickly than traditional, but they will display on their surfaces a greater population of intact heterologous fusion peptides or polypeptides.

The coat protein-displayed molecule conjugates are illustrated as being attached to the spore surface, however, in fact the coat protein component reaches inside the spore coat, since the coat proteins participating in the conjugates herein are part of the spore coat.

The spores displaying the desired molecules, e.g. an scFv library, can be selected by a variety of methods, including magnetic bead selection. Magnetic separation is based on attaching an affinity group to the surface of a magnetic particle. A suspension of such particles is thoroughly mixed with a preparation of the appropriately labeled target conjugate. After an incubation period, during which the labeled target binds to the affinity ligand, a powerful magnet is used to immobilize the magnetic particles and their trapped analytes. The unbound material can be removed by aspiration and the bound material washed and detected. Several protocols incorporate methods to detach the trapped analyte from the bead. Magnetic separations subject analytes to very little mechanical stress compared to other methods, they are rapid, often highly scalable, low cost, and they avoid hazardous or toxic reagents. Thus, separation can be performed using paramagnetic anti-biotin beads (Miltenyi Biotec, Auburn, Calif.) according to the manufacturer's instructions. Spores displaying antibodies or antibody fragments on their surface are magnetically labeled with the corresponding antigen conjugates to magnetic nanospheres (MicroBeads). The spores labeled with the antigen-conjugated MicroBeads are retained on a magnetic column, and the retained spores are eluted as the enriched, positively selected cell fraction.

Another method suitable for the selection of spores displaying the desired fusion is Fluorescencence Activated Cell Sorting (FACS), where a heterogeneous population of suspended spores is characterized and separated based upon the intensity of fluorescence they emit while passing single file through an illuminated volume. Protocols for this method are well known in the art.

The coat protein conjugates of the present invention can also be used to further optimize the coat protein sequences for use in further fusions. Thus, various coat protein fragments, with N- and/or C-terminal deletions, or other variants, containing one or more amino acids substitutions, deletions and/or insertions, can be displayed on the surface of a spore and selected as described above, and tested individually or mixed together, for a desired property, such as antigen binding, to be optimized. Linkers can be optimized in a similar manner.

The coat protein conjugates herein can also contain two or more repeats of the same or different coat protein sequences. Such repeat structures are expected to offer certain advantages over structures comprising a single coat protein sequence, including, for example, increased stability and/or improved resistance to harsher wash conditions.

The location of insertion, or fusion, of the heterologous molecule (e.g. protein) into the carrier coat protein is an important factor, since it can influence the stability, the activity and post-translational modification of the conjugate formed. Therefore, conjugates at the N-terminus, C-terminus or/or interior of the coat protein sequence can be constructed with the same heterologous molecule (e.g. protein) to obtain a more efficient display. However, although interior fusions are possible, N- and C-terminal fusions to a coat protein sequence, such as a Bt coat protein sequence, are preferred. Particularly preferred fusions are N- and C-terminal fusions of the CotB1 and CotB2 sequences disclosed herein (SEQ ID NOS: 1 and 2, respectively), or functional fragments thereof. Other preferred fusions include N- and C-terminal fusions of the CotB1 and B2 sequences shown in FIGS. 10 and 17, and functional fragments thereof.

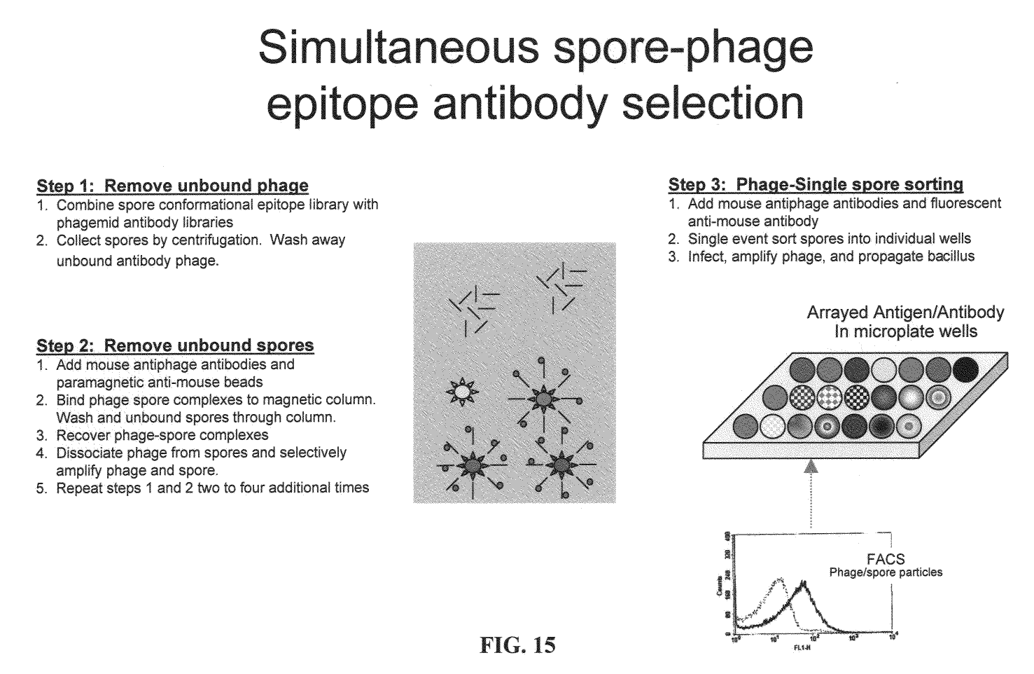

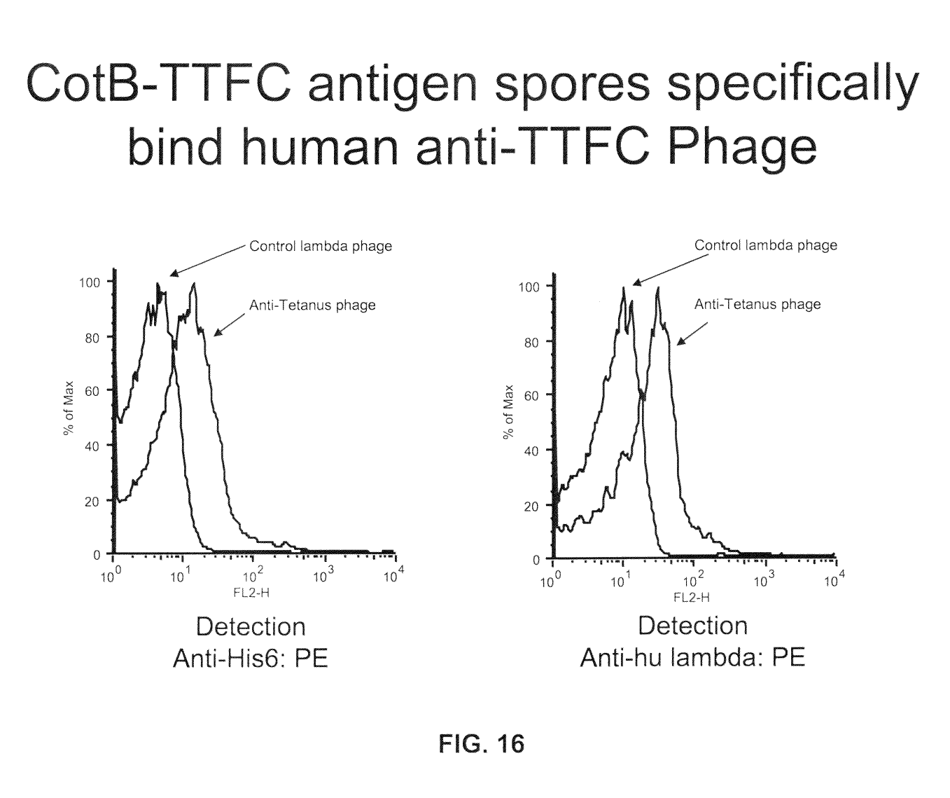

The spore display methods of the present invention can be used for the simultaneous selection of libraries of potential binding partners, such as an epitope library and an antibody library. The simultaneous selection of an epitope library displayed on spores and a phage-display of an antibody library is illustrated in FIG. 15. In brief, in step 1, the spore-displayed conformational epitope library is combined with the phagemid antibody library. The spores are collected by centrifugation, and the unbound antibody phage is washed away. Next, the unbound spores are removed by adding mouse anti-phage antibodies and paramagnetic anti-mouse beads. The phage-spore complexes are bound to the magnetic column, which is then washed to remove the unbound spores. Following these steps, the phage-spore complexes can be recovered, the phage can be dissociated from the spores and the phage and spore can be selectively amplified. The foregoing steps can be repeated as needed, usually two to four additional times. In step 3, spores can be sorted into individual microplate wells, for example, by adding mouse anti-phage antibodies and detectably labeled (e.g. fluorescent) anti-mouse antibodies. The phage can then be amplified, and the bacillus (e.g., B. thuringiensis) carrying the spore-displayed sequences, propagated.

Simultaneous selection of libraries of potential binding partners, such as a conformational epitope and an antibody library, is also possible, if each is displayed using the spore display of the present invention, where the two libraries can be labeled using different tags that allow for selection.

Further details of these and similar “library on library” selection methods are provided in co-pending provisional application No. 60/884,832, filed on Jan. 12, 2007, the entire disclosure of which is expressly incorporated by reference herein.

A major advantage of the spore display system of the present invention over other known display systems is the well-documented stability of spores. It is known that spores can be stored at room temperature for extended periods of time without any significant loss in stability. This property is useful for a variety of applications, such as, for example, for using recombinant spores as a heat-stable oral vaccine.

The spores displaying heterologous peptide or polypeptide molecules can be used as a spore surface display system which finds practical applications in a variety of areas, including, without limitation, screening for binding partners of the peptides/polypeptides displayed, drug delivery, vaccine development and delivery of vaccines, and production of active antibodies and enzymes.

Thus, the spore display system of the present invention is an important tool for protein engineering. In a specific embodiment, the displayed polypeptides are antibody fragments, including, without limitation, single-chain antibody molecules. Such antibody fragments include, for example, diabodies; single-chain antibody molecules (e.g. single-chain Fv (scFv) molecules), Fv, Fab, F(ab′)₂, and Fab′ fragments. Antibody spore displays can be used to identify antibodies binding to a specific target antigen, and to engineer and optimize antibodies for specific selected properties, such as binding affinity and/or selectivity.

In another embodiment, the spore display system herein is used to identify and characterize ligands that bind to a target molecule, i.e. receptor/ligand interactions or protein/protein interactions in general.

In a particular embodiment, the spores are used as an encoded support for conjugated collections of molecules, such as small organic molecules. Thus, a small molecule binding protein, such as streptavidin, can be conjugated to the coat protein displayed on the surface of spores, which can be used to capture biotin-labeled small molecules. The spores are encoded to provide unique labels, such as a nucleic acid “barcode” that is carried either on a replicative or an integrative plasmid for ready coding or decoding of each spore clone. The resulting coded spores can be used in a homogenous selection process to identify and distinguish the biotin-derivatized small molecules. Thus, following particle selection, clonal regrowth, DNA rescue, the barcodes are sequenced, and the immobilized molecules are identified.

It is, of course, possible to use binding interactions different from the streptavidin-biotin interaction, and other barcoding schemes. A similar approach can be used with discrete collections of proteins, cDNA gene products, soluble antibodies, etc.

In addition, the spore display system of the present invention finds utility in vaccine development, for example, as a tool for epitope mapping of antigenic determinants of a virus, such as an HIV or hepatitis C virus, or a bacterium, such as Pseudomonas aeruginosa, a major causative agent of airway infections. The spore display system of the present invention can also be used to select drug candidates, such as peptides, mimicking neutralization epitopes on an infectious agents, such as virus, which can be used in vaccine development. Finally, the spore display system herein can be used as a vaccine delivery vehicle. Thus, engineered spores expressing a heterologous antigen can be used for protective immunization, including oral and trans-mucosal (e.g. nasal) delivery routes.

Further details of the invention are illustrated by the following non-limiting examples. All Bacillus strains used in the examples were obtained from the Bacillus Genetic Stock Center (BGSC).

### EXAMPLE 1

### Recombinant CotB/GFP Protein Fusion Constructs and Expression in Bacillus thuringiensis

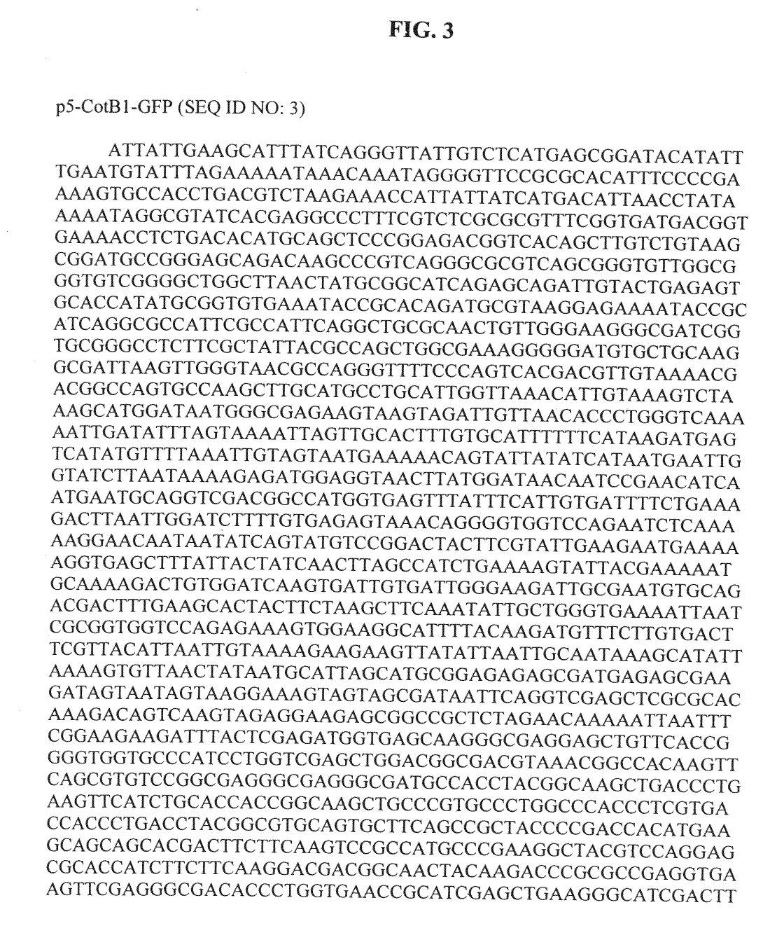

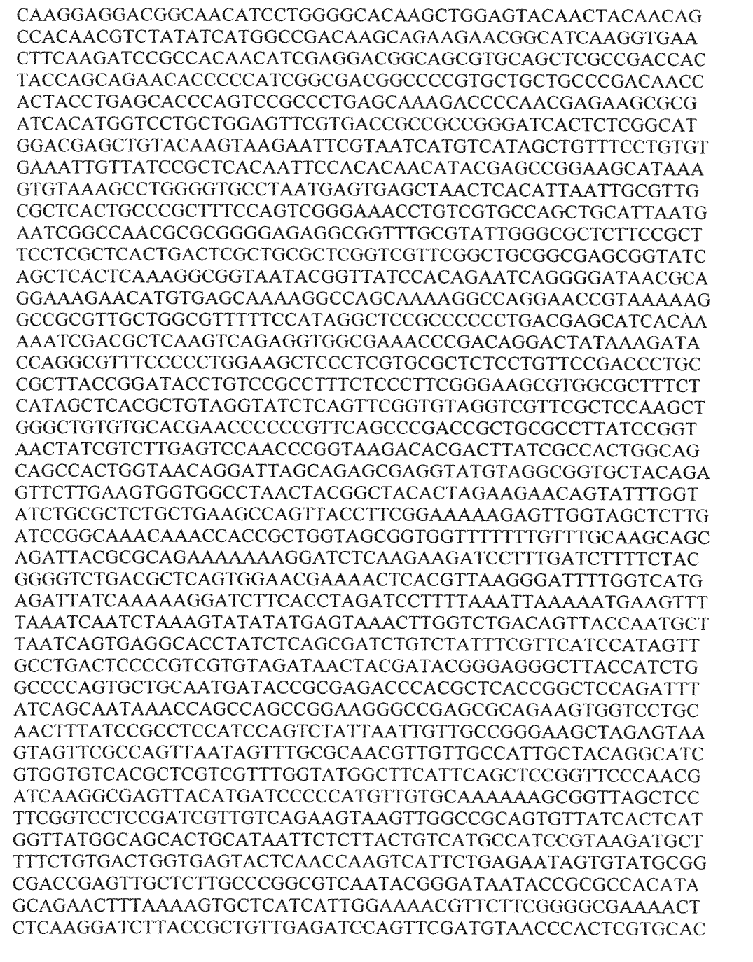

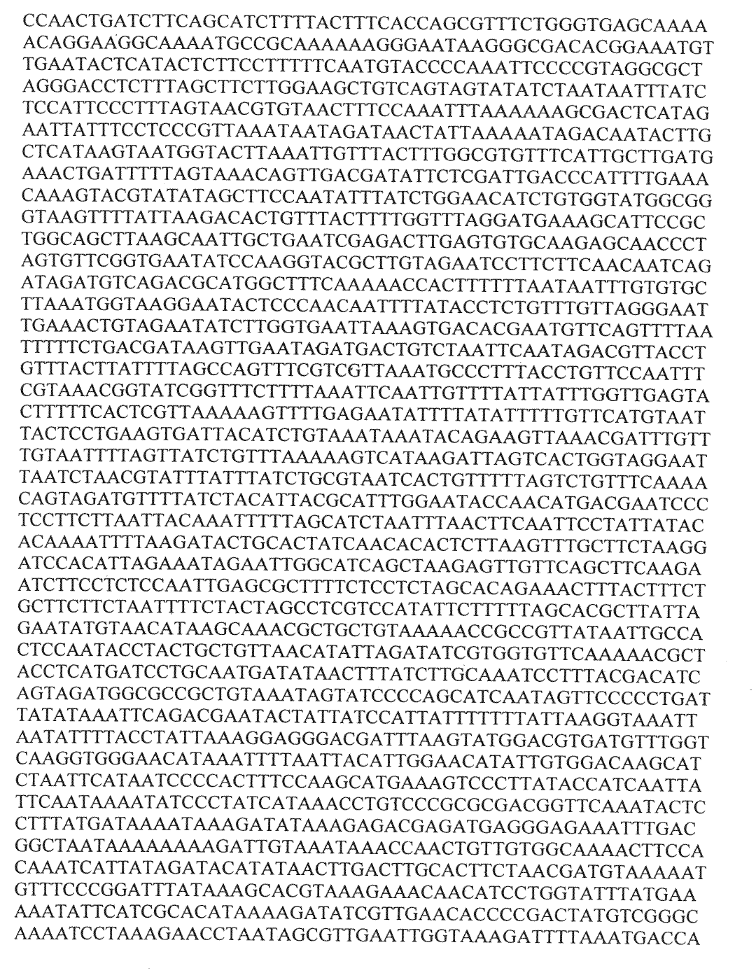

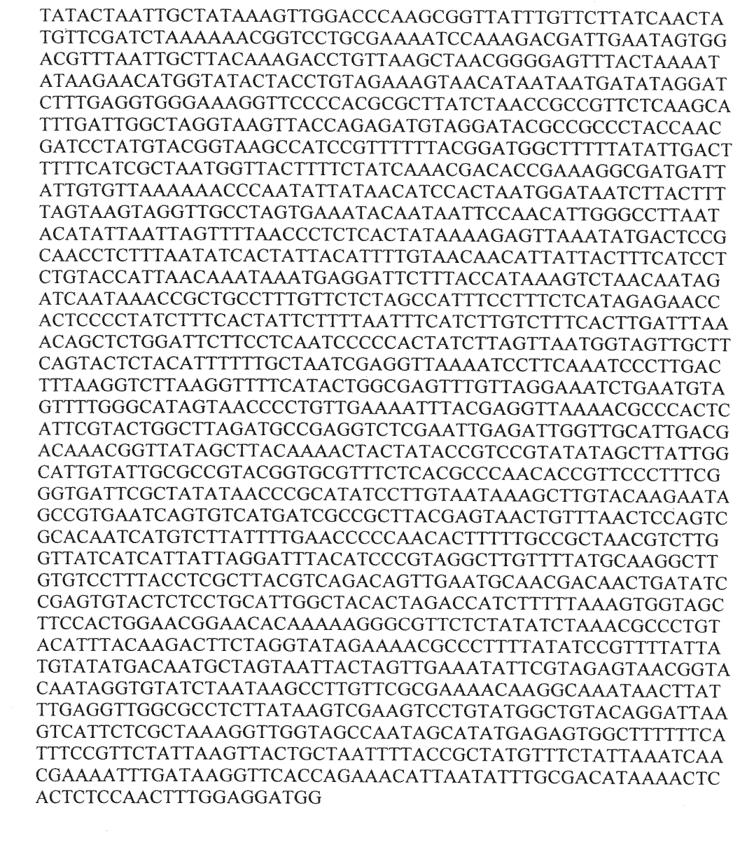

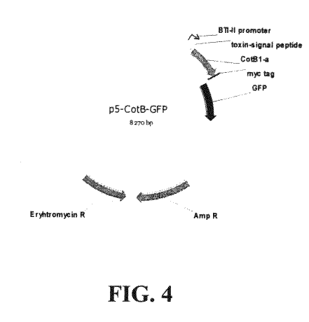

The main construct, p5-CotB1-GFP (FIG. 3, SEQ ID NO: 3), contains a sporulation specific promoter (BtI-II) from the crystal protein Cry1Ac (coat protein) of Bacillus thuringiensis (Bt) followed by the first eleven amino acids of the Cry1A toxin and the CotB1 gene (FIG. 9, SEQ ID NO: 6). For analytical purposes, a myc tag was inserted between the coat protein and the Green Fluorescent Protein gene (GFP). The p5-CotB1-GFP construct also contains an ampicillin resistant gene for selection in Escherichia coli and an erythromycin gene for selection in Bt, and is graphically illustrated in FIG. 4. CotB1 was obtained by amplification by Polymerase Chain Reaction (PCR) of genomic DNA from the a crystalliferous strain of Bt 4D7. Genomic DNA was obtained by lysis of a colony in water followed by boiling for 5 minutes. For cloning purposes PCR primers

| | |
|---|---|
| forward: | |
| CCATGGTGAGTTTATTTCATTGTG | (SEQ ID NO: 11) |
| and | |
| reverse: | |
| GCGGCCGCTCTTCCTCTACT | (SEQ ID NO: 12) |

contained restriction sites for NcoI and NotI.

PCR was performed with the high fidelity Pfx polymerase (Invitrogen) according to manufacturer recommendations. CotB2 (FIG. 9, SEQ ID NO: 7) was obtained by direct DNA synthesis from DNA2.0 using Bacillus optimized codons and also cloned into p5-GFP using the same restriction sites.

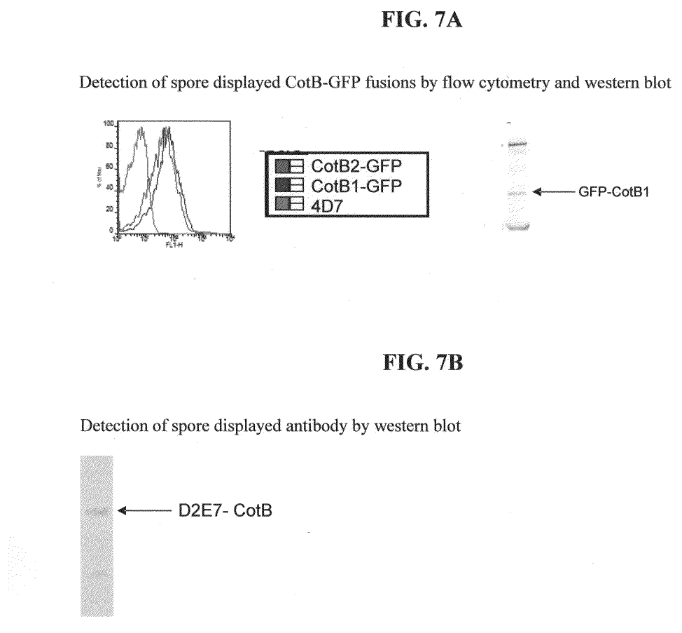

The resulting constructs were propagated in the E. coli strain DH5α (Invitrogen) to verify the sequences, and then transferred to strain JM110 for the production of unmethylated DNA to transform into a crystal minus (4D7) strain of Bt (Bacillus Genetic Stock Center (BGSC)) by electroporation as described (Macaluso, Journal of Bacteriology, 1991, supra). Following outgrowth in Brain Heart Infusion-0.5% Glycerol, the transformants were selected on LB agar plates supplemented with 25 μg/ml erythromycin and incubated overnight at 30° C. Single colonies were grown in GYS sporulation medium (0.2% yeast extract, 0.2% (NH₄)₂SO4, 0.5% K₂HPO₄, 0.1% glucose, 0.002% MgSO₄, 0.0008% CaCl₂, 0.0005% MnCl₂) for 16-20 hours at 30° C. to promote sporulation. An aliquot of the cultures was analyzed for particle distribution by flow cytometry. Typically, 1-15% of spores are free from mother cells. However, spores can be readily liberated following centrifugation and lysis using BPER II (Pierce) supplemented with protease inhibitors for 1 hour at room temperature. This step is important for producing high quality spores with intact non-degraded surface expressed antigens. Finally, the spores were washed 5 times in ice cold sterile distilled water and the presence of spore GFP was confirmed by their fluorescence in flow cytometry and reactivity in western blot analysis (FIG. 7A). For western blot analysis the spore coat proteins must be extracted in 8M urea prior to SDS-PAGE and transfer. The recombinant fusion proteins can be detected by western blot by either of two methods; myc eptitope detection or GFP western analysis.

### EXAMPLE 2

### Spore Surface Display of an Anti-TNF-α Antibody

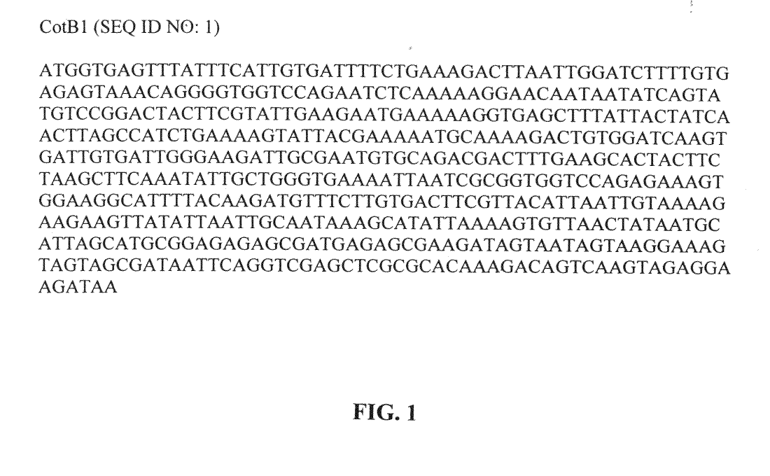

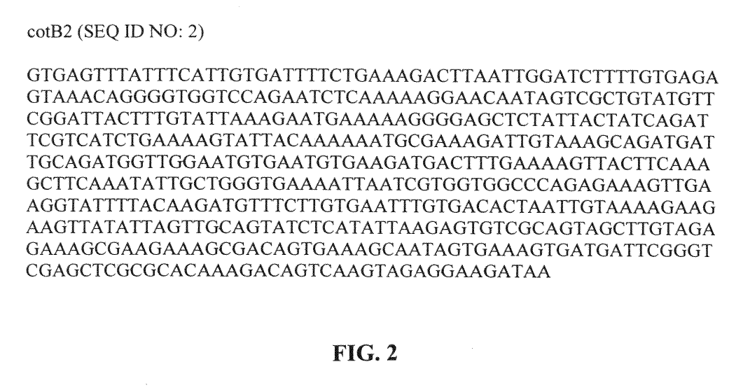

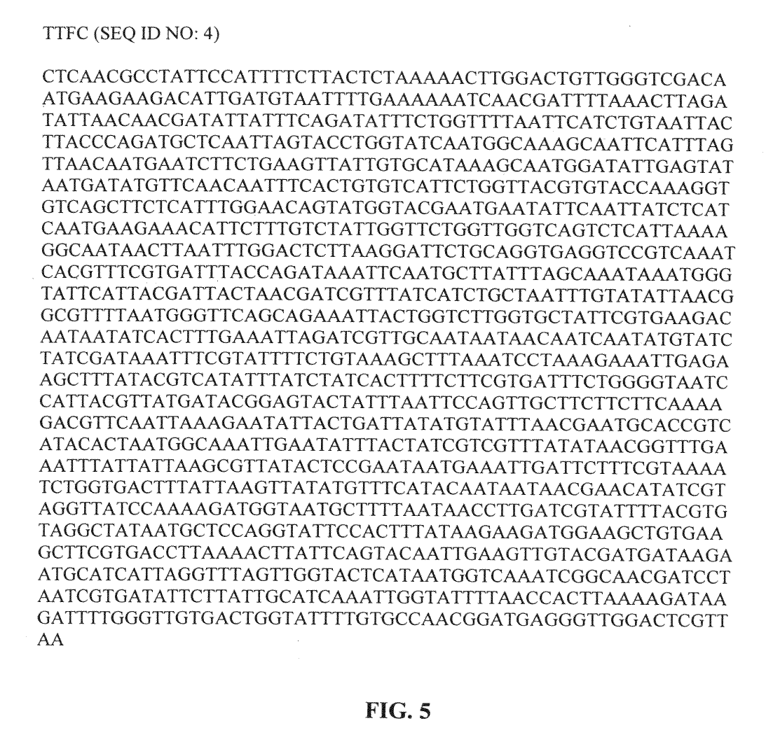

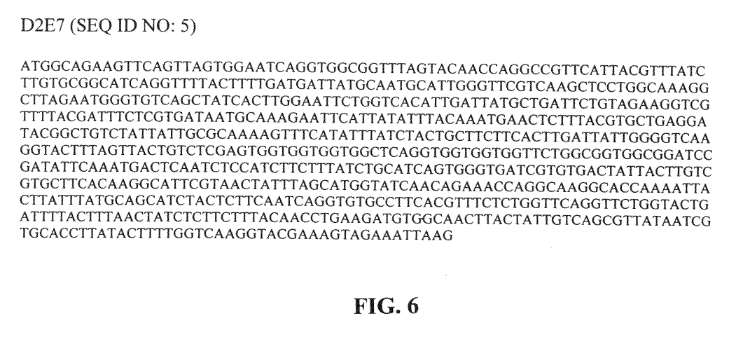

A scFv construct of the anti-human TNF-α antibody D2E7 (FIG. 6, SEQ ID NO: 5) was cloned as an in-frame fusion to the C-terminus of the coat protein Cot B1 (FIG. 1, SEQ ID NO: 1). Spores were obtained as described in Example 1. Fifty microliters of spores were resuspended in FACS blocking buffer (Phosphate Buffered Saline+0.5% Bovine Serum Albumin) for 10 minutes at 4 C. Spores were washed in FACS buffer and then incubated with Phycoerythrin conjugated Streptavidin for 30 minutes at 4 C, washed and then resuspended in PBS. The resulting spores were next analyzed by western blot (FIG. 7B).

### EXAMPLE 3

### Magnetic Bead Selection of scFv SPORE

1×10⁹ washed and blocked spores prepared as described in Example 1 were incubated at 4 C with biotinylated human TNF-α (final concentrations of 50 nM) in a final volume of 1 ml. After two hours, the spores were pelleted, washed 3× with 3 ml PBS and then incubated for 1 hour in 1 ml in the presence of paramagnetic anti-biotin beads (Miltenyi). The bead spore mixture was later applied to a magnetic column, washed, eluted, according to manufacturers instructions, and the eluted spores repropagated as a vegetative cell culture in Brain-Heart Infusion media containing 25 μg/ml erythromycin overnight at 37° C.

### EXAMPLE 4

### FACS Sorting of scFv Spores

1×10⁹ previously washed and blocked spores prepared as described in Example 1, are incubated in 1 ml with 400 nM biotin-TNF-α for 3 hours at room temperature under shaking. Spores are washed with 2 volumes of PBS, then resuspended in PBS/0.5% BSA and labeled with Streptavidin PE (2 mg/ml) and anti-HIS-FITC (25 nM) for 30 minutes on ice, washed and resuspended as previously described.

### EXAMPLE 5

### Spore Surface Enzyme Display

The enzyme β-galactosidase is cloned as an inframe fusion to the cotB1, similarly to previous examples. Spores were obtained as described in Example 1. They are washed and then blocked in PBS+0.5% BSA for 15 minutes on ice and next the fluorogenic substrate fluorescein di-β-D-galactopyranoside (FDG) is incubated with the spores for 20 minutes on ice in the dark and subjected to flow cytometry following a brief PBS wash step.

### EXAMPLE 6

### Spore Immunization and Toxin Challenge

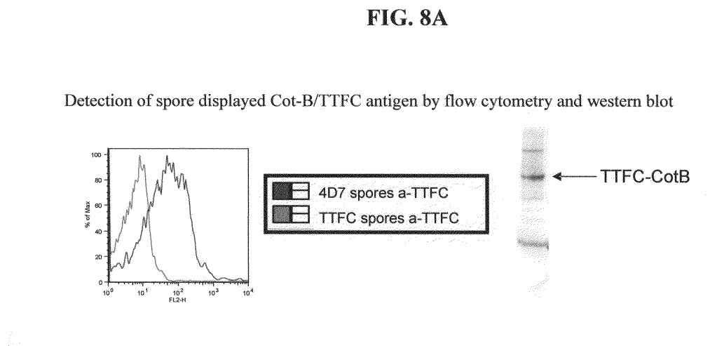

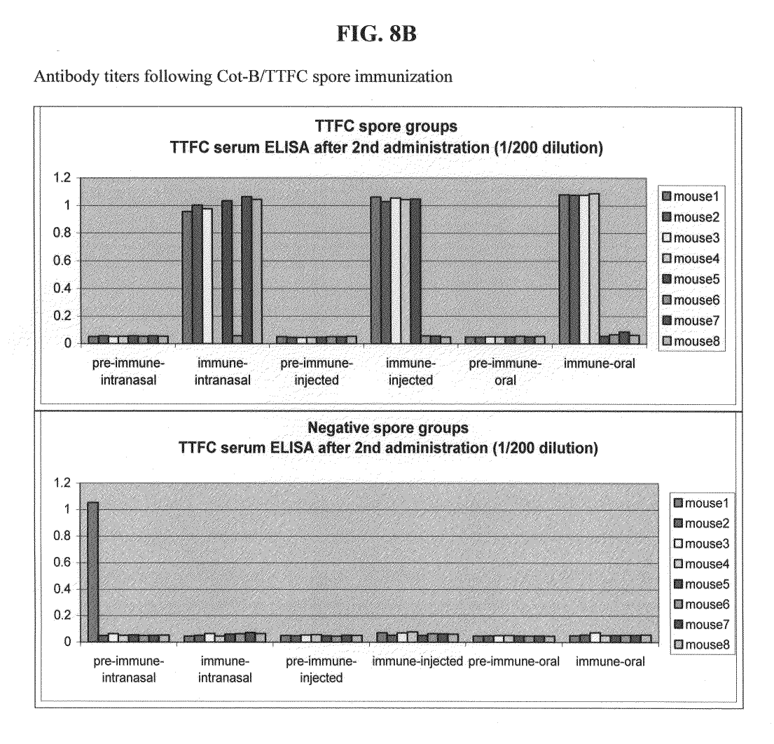

The Tetanus Toxin Fragment C (TTFC) gene (FIG. 5, SEQ ID NO: 4) was synthesized using Bt optimized codons and cloned as in frame fusions to the C-terminus of cotB1 (FIG. 1, SEQ ID NO: 1), similarly to GFP described in Example 1. Expression of TTFC on the surface of the spores was verified by flow cytometry. Briefly spores for immunization were produced as described in Example 1 and an aliquot was analyzed for surface expression. Prior to immunization, the spores were examined for their surface expression of TTFC by flow cytometry. For this analysis an aliquot of spores was incubated for 60 minutes at 4 C with 60 μg/ml mouse anti-TTFC antibody (Roche) in blocking buffer (PBS+0.5% BSA) spores were then washed with ice cold sterile PBS and then resuspended in Phycoerythrin-conjugated anti-mouse antibody. The spore suspension was than incubated on ice for 30 minutes, washed, resuspended and analyzed by flow cytometry (FIG. 8).

For immunization spores were prepared as above, washed 3 times in ice cold sterile water and resuspended to 1E8/ml. Next, groups of eight mice (female, C57 BL/6, 8 weeks) are administered either purified recombinant TTFC (1 μg/mouse), TTFC spores (1E7/mouse), or parental spores (1E7 of 4D7/mouse) orally (0.1 ml), intranasally (0.1 ml), or by intramuscular and/or subcutaneous injection (0.1 ml total/mouse). The administration occurs on days 0, 14, and 28. Serum and feces samples are collected on days −1, 13, 27, 35, and 42.

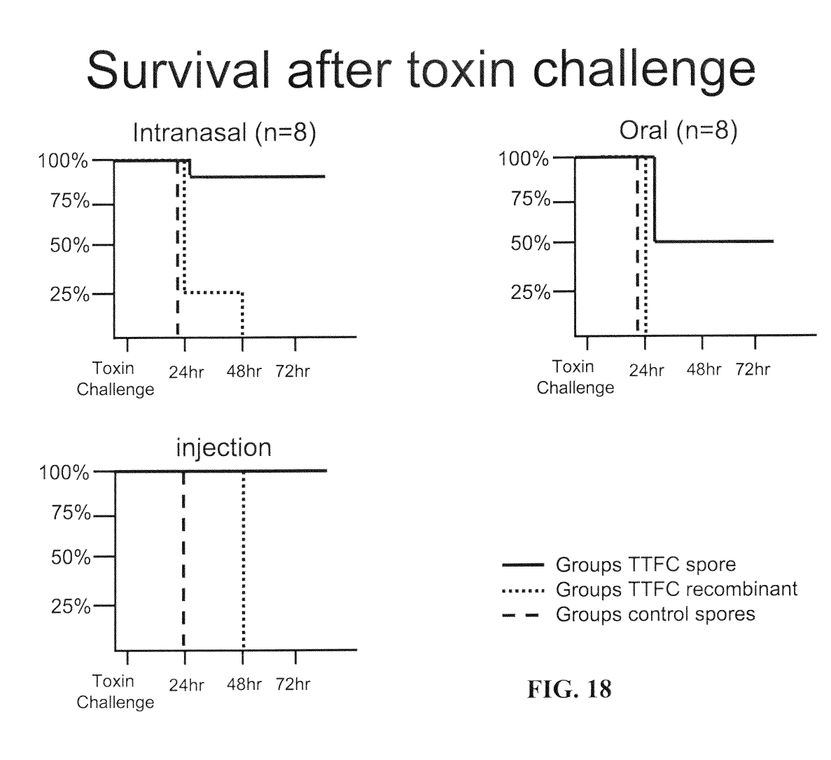

Measurement of immune response is carried out by ELISA. Briefly, plates are coated with 100 μl (2 μg/ml) per well of the specific antigen in coating buffer (BioFx) and left at room temperature overnight. Antigen is recombinant purified TTFC (tetanus toxin fragment C). After blocking with 1% BSA in PBS for 1 hour at room temperature, serum samples are applied using in a dilution series starting in ELISA diluent buffer (0.1 M Tris-HCl, pH 7.4; 3% (w/v) NaCl; 0.5% (w/v) BSA; 10% (v/v) Triton X-100; 0.05% (v/v) Tween-20). Every plate carried replicate wells of a negative control (similarly prepared pre-immune serum at lowest dilution), a positive control (serum from mice immunized parentally with TTFC). Plates are next incubated for 2 hours at room temperature before addition of anti-mouse HRP conjugates (Jackson ImmunoResearch). Plates are incubated for a further 1 hour at 37° C., then developed using the substrate TMB (3,3′,5,5′-tetramethyl-benzidine; BioFX). Reactions are then terminated using ELISA stop solution (BioFX) and their absorbance 495 nm readings recorded. (FIG. 8B) Dilution data are examined in excel and statistical comparisons between groups are made by the Student t-test. A p-value of >0.05 was considered non-significant. For ELISA analysis of fecal IgA, approximately 0.1 g fecal pellets are suspended in 1 ml PBS with 1% BSA and 1 mM PMSF, incubated at 4° C. overnight and then stored at −20° C. prior to ELISA. For each sample, the end-point titer was calculated as the dilution producing the same optical density as the undiluted pre-immune fecal extract. Endpoint titers are reported in FIG. 18

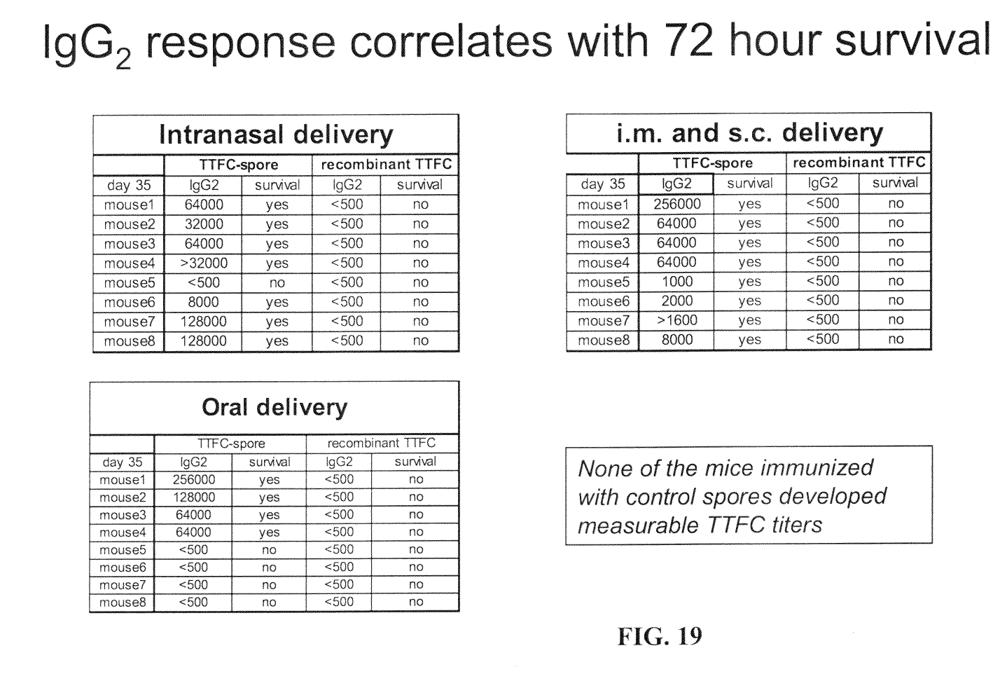

On day 55 all mice were injected sc with 100 ng of tetanus toxin (Calbiochem #582243) in 0.1 ml PBS. Survival was monitored over a 72 hour period and surviving mice were terminated by humane means and blood serum samples collected. Survival curves are reported in FIG. 19.

### EXAMPLE 7

### Tandem (Multiple) Coat Display Constructs

As coat proteins are repeated numerous times to comprise the spore coat, a tandem or multimerized component can strengthen the observed association with the spore particle. In this instance, the coat protein or coat protein fragment is cloned inframe with itself and allows an intervening flexible linker to accommodate any spatial constraints. Thus, a scFv was fused to a twice (or multiply) repeated coat protein or minimized fragment repeat to test whether this display has increased abilities to withstand preparatory or analytical washes and therefore a competitive advantage to its monomeric counterpart. One advantage is that the construct shows increased tolerance to the more stringent washing conditions and treatments required to remove exosporiums from spore particles. In addition, this construct has additional advantages to displayed helical proteins requiring TFE for structural stabilization.

### EXAMPLE 8

### Modular Monomer Fusion Display

The C-terminal coat protein or optimized variant is further engineered to incorporate features beneficial to protein expression, screening and display. For instance, proteolytic substrate sequences, such as the TEV (tobacco etch virus) protease substrate site ENLYFQG (SEQ ID NO: 8), are incorporated between the displayed fusion peptide or polypeptide and the coat protein. In such a construct a gentle and selective proteolytic release from a spore pellet can be used to generate substantial quantities of fusion protein. This proteolytic susceptibility also allows for selective release of the fusion binders, leaving behind nonspecifically bound spore binders. In this instance a fusion of a single peptide, protein epitope, or intact protein to the coat protein is used, and the resulting spores are used to biopan a combinatorial phage antibody library. Phage bound to the spore surface display is recovered by low pH elution or is instead selectively eluted through proteolytic release using the TEV protease.

### EXAMPLE 9

### Modular Multimer Fusion Display

The C-terminal coat protein or optimized variants are engineered to display and coexpress protein multimers. These can be coat protein fusions that form homomeric and heteromeric multimers by opportunistic proximity. A representative example of a homomeric dimer is the erythropoietin receptor, which can dimerize by proximity of two coat protein fusion proteins. An example of heterodimeric proteins occurs if the light chain and heavy chain of an antibody Fab fragment are coexpressed as coat protein fusions and form cognate antigen binding capabilities.

In other instances, coat protein fusions to only one component of the multimer are used and a naturally occurring multimerization is allowed to occur. In this case, to produce a Fab fragment, the heavy chain variable region can be anchored to the coat protein and a freely soluble light chain coexpressed such that the two chains are allowed to assemble through naturally occurring associations within the sporulating cell.

Some multimers that are stabilized through protein-protein interactions unattainable in a spore display system can be forced to assemble through a fusion of associative sequences such as those found in the leucine zippers of fos and jun, or peptide Velcro. An example here is the recreation of an antibody Fv fragment, which is a heterodimer of the variable regions of the heavy and light chains. This is accomplished by fusing the VH region to a fos peptide that is anchored to the coat protein and coexpressing the VL region of an antibody to a complementary jun peptide. As a result, the VH-VL interaction is stabilized to a degree sufficient for screening. This obviates the need for the typical polypeptide linkers that are usually used to constrain the two fragments, but which often greatly alter antigen binding in the scFv format.

Finally, synthetic multimerization is useful when the coat protein-compliment anchors a compliment target fusion through covalent or noncovalent means. In the case of covalent association, disulfide bonds can be formed between the two interacting subunit cysteines such that mild reduction by DTT treatment releases the coat protein associated partner protein. Alternatively, multimers can be assembled through noncovalent means and the complementary association is chosen to be pH sensitive. One such example is the use of a heterodimeric leucine zipper design (Peptide Velcro O'Shea, Current Biology 3(10):658-67 (1993)) which is destabilized by non-neutral pH. In either case, if the target protein is biopanned on the spore surface, and mild reduction or pH-based elution provides a very selective elution scheme.

### EXAMPLE 10

### Spore Conjugates

In this example spores are used as an encoded support for conjugated collections. In such an instance, a small molecule binding protein, such as streptavidin, is fused to the coat protein display and used to addressably immobilize a collection of biotin derivatized small molecules. These spores are encoded to provide unique labels corresponding to large addressable chemical libraries that are discretely immobilized individually to the spore surfaces. The resulting coded spores are then used in a homogenous selection process. Following this selection the best candidates are identified by deconvolution of the encoded spore.

An encoding scheme we have selected is a nucleic acid “barcode” that is carried either on a replicative or integrative plasmid for ready coding and decoding of each spore clone. Following particle selection is clonal regrowth, DNA rescue, and barcode sequencing. As a specific example, any of the four nucleotides are incorporated at three bases in a plasmid, resulting in 64 unique combinations to use as discrete tags. However, this approach is not limited to the use of three bases. By using four bases, the number of the possible unique combinations can be greatly increased.

These addressable libraries can be used with discrete collections of proteins, cDNA gene products, or even soluble antibodies.

### EXAMPLE 11

### Selection of Improved Spore Forming Cells

Towards the end of sporulation the mother cell wall deteriorates and ultimately loses sufficient integrity to isolate the formed spore from the external environment. This outer membrane, or exosporium, if not fully detached from the newly formed spore, can pose a problem for spore surface access and particle uniformity. Restricted access to the spore surface will compromise homogeneous selection. Additionally, heterogeneous particles will have heterogeneous light scattering properties that can confound flow cytometry based analysis and selection. In some cases washing conditions of the spores will reduce the levels of exosporium found in the spores. However, there are likely genetic factors that influence exosporium association. Exosporium-free mutant strains are expected to increase the performance of the display and selection.

Genetic mutants lacking exosporium can arise spontaneously, or a culture of bacilli can be exposed to UV to promote genetic mutation or chemically mutagenized. Exosporium-free spore mutants have a higher density (1.380-1.400 versus 1.340) and are less hydrophobic than wild type spores. These differential properties can be used to isolate exosporium mutants of increased density by a sodium bromide gradient or those with decreased hydrophobicity in a hexadecane partitioning method. The corresponding spore loss of the exosporium can be physically monitored by crystal violet staining and phase contrast microscopy, or quantitatively measured by scatter measurements from flow cytometry analysis.

### EXAMPLE 12

### Selection of Spore Mutants with Improved Display Stability

During sporulation numerous genes are translated, including proteolytic enzymes, some of which have detrimental effects upon heterologous displayed proteins. To address this mutants lacking opportunistic and detrimental proteases are searched for or created. Protease-free mutant strains are expected to increase the performance of the display and selection. Genetic mutants lacking proteases can arise spontaneously, or a culture of Bacilli can be exposed to UV to promote genetic mutation or be chemically mutagenized. Protease-free spore mutants have more stably displayed heterologous proteins and can therefore be isolated by increased display or even by the presence of displayed proteins under conditions where they normally have lost such displayed proteins. For instance at 16 hours GFP spores still have intact fluorescence, but it is lost after an additional 24 hours. The most fluorescent spores are clonally isolated at 16 hours or those maintaining fluorescence after 40 hours of sporulation by flow cytometry.

### EXAMPLE 13

### Selection for Tetanus Toxin Binding Proteins

1×10⁸ washed and blocked tetanus toxin fragment C (TTFC) spores were prepared essentially as described in Example 1 were incubated at 4 C with 1×10¹⁰ cfu of phagemid derived from an human monoclonal antibody that binds tetanus toxin, in a final 1 ml volume. After two hours, the spores were pelleted, washed 10× with 1 ml PBS. A portion of the spore phage complexes was then examined by flow cytometry to monitor specificity of binding (FIG. 16). However, following the aforementioned washes the remaining spore phage complex was incubated with 0.05 ml Tris-buffered 0.2 M glycine (pH 2.2) for 10 minutes, neutralized with 0.008 ml 2M Tris base, and then clarified by centrifugation. This neutralized and clarified phage containing elution was then used to infect E. coli and the titer of recovered phage determined by propagation and limiting dilution on LB-ampicillin agar plates. The quantities of recovered phage from TTFC spores and underivatized parental 4D7 spores were then analyzed for their ability to enrich the TTFC binding antibodies. In this example a monoclonal phage is used but, the same procedure could be used to select from a collection of naïve phage antibodies or from an appropriately enriched collection of antibody phagemids. Furthermore, one could naturally extend this selection to a library against library selection. Specifically, by mixing a collection of spore displayed antigen polypeptides, as those found in antigen libraries, with a phage antibody library, one could simultaneously select for antigen antibody partners.

### EXAMPLE 14

### Generation and Selection of Optimized Coat Proteins

In this example we begin by using error-prone PCR to create a collection of coat protein variants. The variants are then cloned in frame with GFP. Following this cloning the collection is transferred into Bacillus and then sporulated as previously described. Following 16-48 hours of sporulation the collection is subjected to flow cytometry. Variants with improved stability and expression will remain fluorescent and will be bulk sorted to select those with the greatest fluorescence. The flow cytometry selection would be repeated to reinforce selection of those desired clones with optimized coat proteins. Finally, the resulting bulk sorted variants could then be clonally sorted by limiting dilution or by flow cytometry.

### EXAMPLE 15

### Improving Spore Protein Display Through Toxin Based Amino Terminal Peptides

Bacillus delta toxins are robustly produced as protoxins that are ultimately cleaved and activated by removal of a small region located at the amino terminus approximately 28-29 amino acids from the start codon. The toxins can accumulate during sporulation to levels that constitute up to 25% of the total protein mass of the resulting Bacillus Thuringiensis spore. The use of the spore specific promoter region that precedes endogenous toxin genes facilitates high level and spore specific heterologous protein expression. However, the amino terminus of the toxin has high translation level potential that could benefit heterologous recombinant protein expression.

Different toxin amino acid sequences also have potential to increase overall heterologous protein production. To test for net protein increase on the spores cotB:GFP fusions are generated with differing amino-termini from a collection of other Bacillus Thuringiensis strains and assess directly their spore specific recombinant protein content by flow cytometry.

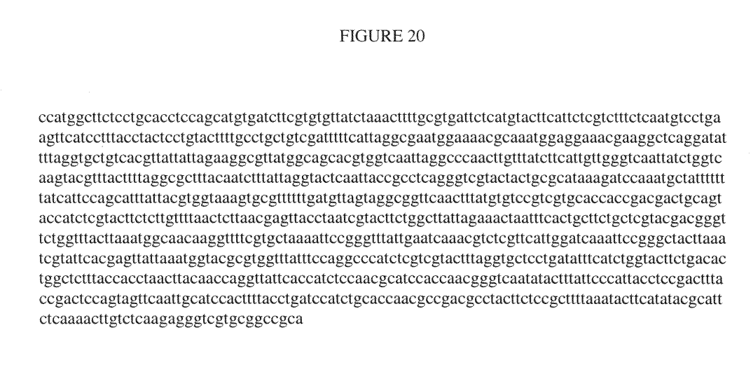

For example DNA corresponding to the amino termini listed in FIG. 20 (SEQ ID Nos: 46-53) is synthesized, and all possible chimeras to cotB:GFP are generated. The resulting spores are examined for fluorescence and the best clones with highest fluorescent signal selected for further work.

Alternatively or in addition, it is possible to design and introduce, via combinatorial peptide library, a small diversified collection of sequences to utilize for optimization. In this instance a combinatorial peptide library is designed and made based upon any of the naturally occurring the range of toxin sequences found, their consensus sequences, or randomly and then FACS sort those spores with the highest fluorescence.

### EXAMPLE 16

### Spore Surface Display of a Small Peptide

During sporulation the vegetative cell functionally compartmentalizes to form an endospore. Concurrent with the process of spore formation the remaining vegetative cell is converts to and is exposed to an overall catabolic environment. In this catabolic environment one would expect small peptides to be proteolytically unstable. Fusion of small peptides to large proteins such as the toxin or cotB protein could confer stability to such recombinant peptides. To explore this possibility we cloned small recombinant peptides to cotB and analyzed their expression on the surface of resulting spores

Specifically, the first fifteen amino acids from the amyloid beta polypeptide (LEDAEFRHDSGYEVHHQ) (SEQ ID NO: 54) was cloned as an in-frame fusion to the carboxy-terminus of the coat protein Cot B1 and transformed into Bacillus Thuringiensis (FIG. 1, SEQ ID NO: 1). The resulting transformants were then sporulated as described in Example 1. Fifty microliters of the spores were resuspended and blocked in FACS blocking buffer (Phosphate Buffered Saline+0.5% Bovine Serum Albumin) for 10 minutes at 4 C and then incubated with 50 ug/ml of mouse monoclonal (2C8) antibody to beta amyloid for one hour at 4 C. Spores were next washed in FACS buffer and incubated with Phycoerythrin conjugated anti-mouse kappa chain for 30 minutes at 4 C, washed and resuspended in PBS. These resuspended spores were next analyzed by flow cytometry FIG. Y.

### EXAMPLE 17

### Spore Surface Display at the Amino Terminus of CotB

Recombinant protein fusions are useful and convenient expression tools to produce proteins of interest. However, free amino termini are sometimes necessary for proper function. In this regard we cloned and expressed a protein as a fusion to the amino terminus of the CotB protein.

Specifically, we synthesized DNA corresponding to the mature Thrombopoietin (Tpo) gene product using Bacillus thuringiensis optimized codons human (Tpo) (FIG. 20; SEQ ID NO: 55) and cloned it as an frame fusion the amino terminus of CotB1 (FIG. 1, SEQ ID NO: 1). The resulting transformants were then sporulated as previously described in Example 1. Fifty microliters of the spores were resuspended and blocked in FACS blocking buffer (Phosphate Buffered Saline+0.5% Bovine Serum Albumin) for 10 minutes at 4 C and then incubated with 50 μg/ml of anti-myc antibody, whose epitope was present on the carboxy terminus of the CotB protein, for one hour at 4 C. Spores were next washed in FACS buffer and incubated with Phycoerythrin conjugated anti-mouse kappa chain for 30 minutes at 4 C, washed and resuspended in PBS. Expression of Tpo on the surface of the spores was verified by flow cytometry as described previously. The amino-terminal display proved to be efficient for Tpo expression FIG. Z.

Although in the foregoing description the invention is illustrated with reference to certain embodiments, it is not so limited. Indeed, various modifications of the invention in addition to those shown and described herein will become apparent to those skilled in the art from the foregoing description and fall within the scope of the appended claims. Thus, while the invention is illustrated with reference to antibody libraries, it extends generally to all peptide and polypeptide libraries.

All references cited throughout the specification are hereby expressly incorporated by reference.

What is claimed is:

1. A conjugate comprising:
(a) the full-length sequence of an externally exposed native sequence spore coat protein of a Bacillus; or
(b) a functional fragment of an externally exposed native sequence spore coat protein of a Bacillus, other than Bacillus subtilis, or
(c) a functional variant of (a) or (b),
conjugated to a heterologous molecule.

2. The conjugate of claim 1 displayed on the surface of a Bacillus spore.

3. The conjugate of claim 1 or claim 2 wherein the heterologous molecule is a peptide or a polypeptide.

4. The method of claim 1 or claim 2 wherein the heterologous molecule is an antibody or an antibody fragment, or a surrobody or a surrobody fragment.

5. The method of claim 4 wherein the antibody fragment is an antibody heavy or light chain, or a fragment thereof.

6. The conjugate of claim 1 or claim 2 wherein the conjugate is a direct fusion between the spore coat protein and the heterologous molecule.

7. The conjugate of claim 6 wherein the fusion is between the C-terminus of the spore coat protein and the heterologous molecule.

8. The conjugate of claim 6 wherein the fusion is between the N-terminus of the spore coat protein and the heterologous molecule.

9. The conjugate of claim 1 or claim 2 wherein the heterologous molecule is linked to the coat protein through a linker.

10. The conjugate of claim 9 wherein the linker is a peptide sequence.

11. The conjugate of claim 10 wherein the peptide sequence comprises a substrate sequence for an enzyme.

12. The conjugate of claim 11 wherein the enzyme is a protease.

13. The conjugate of claim 9 wherein the linker is a dimeric linker.

14. The conjugate of claim 13 wherein the dimeric linker comprises a covalent association between two binding partners.

15. The conjugate of claim 14 wherein the covalent association is provided by a disulfide bond.

16. The conjugate of claim 13 wherein the dimeric linker comprises a non-covalent association between two partners.

17. The conjugate of claim 16 wherein the non-covalent association is between a pair of leucine zipper peptides.

18. The conjugate of claim 17 wherein the leucine zipper peptides are selected from the group consisting of c-Jun and v-Fos.

19. The conjugate of claim 1 or claim 2 wherein the Bacillus is selected from the group consisting of Bacillus thuringiensis, Bacillus cereus, Bacillus anthracis, Bacillus amyloliquefaciens, Bacillus weihenstephanensis; Geobacillus kaustophilus; and Geobacillus thermodenitrificans.

20. The conjugate of claim 19 wherein the Bacillus is Bacillus thuringiensis.

21. The conjugate of claim 20 comprising Bacillus thuringiensis CotB1 (SEQ ID NO: 6) or CotB2 (SEQ ID NO: 7), or a functional fragment or variant thereof.

22. The conjugate of claim 1, part (c) or claim 2, wherein the functional variant is a chimeric molecule comprising externally exposed spore coat protein sequences from more than one Bacilli, or more than species or sub-species of the same Bacillus.

23. The conjugate of claim 22 wherein at least one of said Bacilli is Bacillus thuringiensis.

24. The conjugate of claim 22 wherein at least one of said Bacilli is Bacillus subtilis.

25. A nucleic acid molecule comprising a nucleotide sequence encoding the conjugate of claim 1.

26. A nucleic acid molecule comprising a nucleotide sequence encoding the conjugate of claim 6.

27. A nucleic acid molecule comprising a nucleotide sequence encoding the conjugate of claim 10.

28. The nucleic acid molecule of any one of claims 25 to 27 further comprising regulatory sequences capable of directing the expression of the nucleic acid molecule on a Bacillus spore.

29. The nucleic acid molecule of claim 28 wherein the regulatory sequences comprise a sporulation-specific promoter region.

30. The nucleic acid molecule of any one or claims 25 to 27 comprising a further nucleotide sequence encoding an N-terminal Bacillus peptide preceding the coding sequence of the mature native sequence spore coat protein or a functional fragment or variant thereof.

31. The nucleic acid molecule of claim 28 comprising a further nucleotide sequence encoding an N-terminal Bacillus peptide preceding the coding sequence of the mature native sequence spore coat protein or a functional fragment or variant thereof.

32. A cell of a spore forming Bacillus comprising and capable of expressing a nucleic acid of any one of claims 25 to 27.

33. A cell of a spore forming Bacillus comprising and capable of expressing a nucleic acid of claim 29 or claim 31.

34. A cell of a spore forming Bacillus comprising and capable of expressing a nucleic acid of claim 30.

35. A recombinant sporulating spore forming Bacillus expressing the conjugate of claim 1 on the surface of the spores thereof.

36. The recombinant sporulating spore forming Bacillus of claim 35 wherein the Bacillus is selected from the group consisting of Bacillus thuringiensis, Bacillus cereus, Bacillus anthracis, Bacillus amyloliquefaciens, Bacillus weihenstephanensis, Geobacillus kaustophilus; and Geobacillus thermodenitrificans.

37. The recombinant sporulating spore forming Bacillus of claim 36 wherein the Bacillus is Bacillus thuringiensis.

38. A cell culture comprising cells of the recombinant sporulating spore forming Bacillus of claim 35.

39. A display system comprising a plurality of identical or different conjugates comprising:
(a) the full-length sequence of an externally exposed native sequence spore coat protein of a Bacillus; or
(b) a functional fragment of an externally exposed native sequence spore coat protein of a Bacillus, other than Bacillus subtilis, or
(c) a functional variant of (a) or (b),
conjugated to one or more heterologous molecules.

40. The display system of claim 39 wherein the heterologous molecules present in the conjugates are peptides or polypeptides.

41. The display system of claim 40 wherein the peptides or polypeptides are members of a peptide or polypeptide library.

42. The display system of claim 41 wherein the peptides or polypeptides are structurally related to each other.

43. The display system of claim 41 wherein the peptides or polypeptides are functionally related to each other.

44. The display system of claim 41 wherein the polypeptides are antibodies or antibody fragments, or surrobodies or surrobody fragments.

45. The display system of claim 44 wherein the antibody fragments are selected from the group consisting of Fab, Fab′, F(ab′)₂, scFv, (scFv)₂, dAb, complementarity determining region (CDR) fragments, linear antibodies, single-chain antibody molecules, minibodies, diabodies, and multispecific antibodies formed from antibody fragments.

46. The display system of claim 45 wherein the antibody fragments are scFv fragments.

47. The display system of claim 39 wherein the heterologous molecules are non-peptide small molecules.

48. The display system of claim 39 wherein the conjugates are direct fusions between said coat protein and heterologous molecules.

49. The display system of claim 39 wherein in the conjugates the heterologous molecules are linked to the coat protein through a linker.

50. The display system of claim 49 wherein the linker is a peptide sequence.

51. The display system of claim 50 wherein the peptide sequence comprises a substrate sequence for an enzyme.

52. The display system of claim 51 wherein the enzyme is a protease.

53. The display system of claim 49 wherein the linker is a dimeric linker.

54. The display system of claim 53 wherein the dimeric linker comprises a covalent association between two binding partners.

55. The display system of claim 54 wherein the covalent association is provided by a disulfide bond.

56. The display system of claim 53 wherein the dimeric linker comprises a non-covalent association between two partners.

57. The display system of claim 56 wherein the non-covalent association is between a pair of leucine zipper peptides.

58. The display system of claim 39 wherein at least some of the conjugates comprise multiple copies of the sequence of the coat protein.

59. The display system of claim 39 wherein each of the conjugates comprises the same coat protein sequence.

60. The display system of claim 39 wherein the conjugates comprise different coat protein sequences.

61. The display system of claim 39 wherein the conjugates comprise monomeric units of a multimeric polypeptide.

62. The display system of claim 61 wherein the monomeric units are displayed in a proximity that allows combination of said units to form a multimeric polypeptide.

63. The display system of claim 61 wherein the multimeric polypeptide is a dimeric polypeptide.

64. The display system of claim 62 wherein the multimeric polypeptide is an antibody or antibody fragment and the monomeric units displayed are antibody heavy and light chains or fragments thereof.

65. The display system of claim 39 wherein the Bacillus is selected from the group consisting of Bacillus thuringiensis, Bacillus cereus, Bacillus anthracis, Bacillus amyloliquefaciens, Bacillus weihenstephanensis, Geobacillus kaustophilus; and Geobacillus thermodenitrificans.

66. The display system of claim 65 wherein the Bacillus is Bacillus thuringiensis.

67. The display system of claim 39, part (c), wherein the functional variant is a chimeric molecule comprising externally exposed spore coat protein sequences from more than one Bacilli, or more than species or sub-species of the same Bacillus.

68. The display system of claim 67 wherein at least one of said Bacilli is Bacillus thuringiensis.

69. The display system of claim 67 wherein at least one of said Bacilli is Bacillus subtilis.

70. The display system of claim 68 wherein the Bt coat protein sequence is selected from the group consisting of a CotB1 protein sequence of SEQ ID NO: 6, a CotB2 protein sequence of SEQ ID NO: 7, and functional fragments and variants thereof.

71. The display system of claim 70 wherein the sequence of said variant has at least about 80% sequence identity to the amino acid sequence of SEQ ID NO: 6 or SEQ ID NO: 7, and the conjugates formed are capable of stable association with the surface of Bt spores.

72. The display system of claim 68 wherein the Bt coat protein is from a Bt subspecies selected from the group consisting of Bt kurstaki, Bt dendrolimus, Bt galleriae, Bt entomocidus, Bt aizawai, Bt morrisoni, Bt tolworthi, Bt alesti, and Bt israelensis.

73. The display system of claim 39 wherein at least some of the spores are bar-coded to provide unique labels.

74. The display system of claim 73 wherein the unique label is a nucleic acid barcode generated by combinations of three to 20 nucleotides.

75. A method for displaying a collection of peptide or polypeptides on the surface of spores, comprising expressing said collection of peptides or polypeptides on the surface of spores of a Bacillus in the form of conjugates comprising:
(a) the full-length sequence of an externally exposed native sequence spore coat protein of a Bacillus; or
(b) a functional fragment of an externally exposed native sequence spore coat protein of a Bacillus, other than Bacillus subtilis, or
(c) a functional variant of (a) or (b),
conjugated to said peptides or polypeptides.

76. The method of claim 75 wherein substantially all of the spores are exosporium-free.

77. The method of claim 75 wherein at least about 90% of the spores are exosporium-free.

78. The method of claim 75 wherein the Bt spores are previously selected to be exosporium-free mutants.

79. The method of claim 75 wherein the Bacillus is Bacillus thuringiensis.

80. The method of claim 75 wherein the displayed conjugates are formed by transforming Bacillus with nucleic acid encoding said conjugates, each under control of a sporulation specific promoter, and culturing and harvesting the transformed Bacillus under conditions to support sporulation and stable protein display.

81. The method of claim 80 wherein colonies of the transformed spores are grown in a sporulation medium for less than 48 hours, whereupon the spores are liberated retaining the majority of the displayed peptides or polypeptides in an intact, non-degraded form.

82. The method of claim 80 further comprising the step of testing the stability of the display.

83. The method of claim 80 further comprising the step of testing the chemical or biological integrity of one or more peptides or polypeptides displayed.

84. The method of claim 80 further comprising a step of selecting the Bacillus spores displaying a coat protein-peptide or -polypeptide conjugate.

85. The method of claim 84 wherein the selection is performed by magnetic sorting.

86. The method of claim 84 wherein the selection is performed by flow cytometry.

87. A spore carrying the fusion polypeptide encoded by the nucleic acid molecule of any one of claims 25 to 31.

88. The spore of claim 87 wherein the fusion polypeptide is stably anchored to the spore.

89. The spore of claim 88 wherein the heterologous peptide or polypeptide is displayed on the surface of the spore.

90. The spore of claim 89 wherein the heterologous peptide or polypeptide is biologically active.

91. The spore of claim 90 wherein the heterologous peptide or polypeptide is a therapeutic agent.

92. A vaccine comprising an antigen-Bacillus coat protein conjugate displayed on the surface of a spore.

93. The vaccine of claim 92 suitable for oral administration, transmucosal delivery, or parenteral administration.

94. The vaccine of claim 93 wherein the transmucosal delivery is intra-nasal administration.

95. The vaccine of claim 92 selected from the group consisting of a flu vaccine, a vaccine for childhood immunization, and an HIV vaccine.


SEQUENCE LISTING (55 sequences)

SEQ ID NO: 1 - DNA, 528 bp, Bacillus thuringiensis
atggtgagtttatttcattgtgattttctgaaagacttaattggatcttttgtgagagtaaacaggggtggtccagaatctcaaaaaggaacaataatatcagtatgtccggactacttcgtattgaagaatgaaaaaggtgagctttattactatcaacttagccatctgaaaagtattacgaaaaatgcaaaagactgtggatcaagtgattgtgattgggaagattgcgaatgtgcagacgactttgaagcactacttctaagcttcaaatattgctgggtgaaaattaatcgcggtggtccagagaaagtggaaggcattttacaagatgtttcttgtgacttcgttacattaattgtaaaagaagaagttatattaattgcaataaagcatattaaaagtgttaactataatgcattagcatgcggagagagcgatgagagcgaagatagtaatagtaaggaaagtagtagcgataattcaggtcgagctcgcgcacaaagacagtcaagtagaggaagataa

SEQ ID NO: 2 - DNA, 510 bp, Bacillus thuringiensis
gtgagtttatttcattgtgattttctgaaagacttaattggatcttttgtgagagtaaacaggggtggtccagaatctcaaaaaggaacaatagtcgctgtatgttcggattactttgtattaaagaatgaaaaaggggagctctattactatcagattcgtcatctgaaaagtattacaaaaaatgcgaaagattgtaaagcagatgattgcagatggttggaatgtgaatgtgaagatgactttgaaaagttacttcaaagcttcaaatattgctgggtgaaaattaatcgtggtggcccagagaaagttgaaggtattttacaagatgtttcttgtgaatttgtgacactaattgtaaaagaagaagttatattagttgcagtatctcatattaagagtgtcgcagtagcttgtagagaaagcgaagaaagcgacagtgaaagcaatagtgaaagtgatgattcgggtcgagctcgcgcacaaagacagtcaagtagaggaagataa

SEQ ID NO: 3 - DNA, 8270 bp, Artificial Sequence
  source: /note=“Description of Artificial Sequence Synthetic polynucleotide”
attattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcattggttaaacattgtaaagtctaaagcatggataatgggcgagaagtaagtagattgttaacaccctgggtcaaaaattgatatttagtaaaattagttgcactttgtgcattttttcataagatgagtcatatgttttaaattgtagtaatgaaaaacagtattatatcataatgaattggtatcttaataaaagagatggaggtaacttatggataacaatccgaacatcaatgaatgcaggtcgacggccatggtgagtttatttcattgtgattttctgaaagacttaattggatcttttgtgagagtaaacaggggtggtccagaatctcaaaaaggaacaataatatcagtatgtccggactacttcgtattgaagaatgaaaaaggtgagctttattactatcaacttagccatctgaaaagtattacgaaaaatgcaaaagactgtggatcaagtgattgtgattgggaagattgcgaatgtgcagacgactttgaagcactacttctaagcttcaaatattgctgggtgaaaattaatcgcggtggtccagagaaagtggaaggcattttacaagatgtttcttgtgacttcgttacattaattgtaaaagaagaagttatattaattgcaataaagcatattaaaagtgttaactataatgcattagcatgcggagagagcgatgagagcgaagatagtaatagtaaggaaagtagtagcgataattcaggtcgagctcgcgcacaaagacagtcaagtagaggaagagcggccgctctagaacaaaaattaatttcggaagaagatttactcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgtaccccaaattccccgtaggcgctagggacctctttagcttcttggaagctgtcagtagtatatctaataatttatctccattccctttagtaacgtgtaactttccaaatttaaaaaagcgactcatagaattatttcctcccgttaaataatagataactattaaaaatagacaatacttgctcataagtaatggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgaatgttcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaattactcctgaagtgattacatctgtaaataaatacagaagttaaacgatttgtttgtaattttagttatctgtttaaaaagtcataagattagtcactggtaggaattaatctaacgtatttatttatctgcgtaatcactgtttttagtctgtttcaaaacagtagatgttttatctacattacgcatttggaataccaacatgacgaatccctccttcttaattacaaatttttagcatctaatttaacttcaattcctattatacacaaaattttaagatactgcactatcaacacactcttaagtttgcttctaaggatccacattagaaatagaattggcatcagctaagagttgttcagcttcaagaatcttcctctccaattgagcgcttttctcctctagcacagaaactttactttctgcttcttctaattttctactagcctcgtccatattctttttagcacgcttattagaatatgtaacataagcaaacgctgctgtaaaaaccgccgttataattgccactccaatacctactgctgttaacatattagatatcgtggtgttcaaaaacgctacctcatgatcctgcaatgatataactttatcttgcaaatcctttacgacatcagtagatggcgccgctgtaaatagtatccccagcatcaatagttccccctgattatataaattcagacgaatactattatccattatttttttattaaggtaaattaatattttacctattaaaggagggacgatttaagtatggacgtgatgtttggtcaaggtgggaacataaattttaattacattggaacatattgtggacaagcatctaattcataatccccactttccaagcatgaaagtcccttataccatcaattattcaataaaatatccctatcataaacctgtcccgcgcgacggttcaaatactcctttatgataaaataaagatataaagagacgagatgagggagaaatttgacggctaataaaaaaaagattgtaaataaaccaactgttgtggcaaaacttccacaaatcattatagatacatataacttgacttgcacttctaacgatgtaaaaatgtttcccggatttataaagcacgtaaagaaacaacatcctggtatttatgaaaaatattcatcgcacataaaagatatcgttgaacaccccgactatgtcgggcaaaatcctaaagaacctaatagcgttgaattggtaaagattttaaatgaccatatactaattgctataaagttggacccaagcggttatttgttcttatcaactatgttcgatctaaaaaacggtcctgcgaaaatccaaagacgattgaatagtggacgtttaattgcttacaaagacctgttaagctaacggggagtttactaaaatataagaacatggtatactacctgtagaaagtaacataataatgatataggatctttgaggtgggaaaggttccccacgcgcttatctaaccgccgttctcaagcatttgattggctaggtaagttaccagagatgtaggatacgccgccctaccaacgatcctatgtacggtaagccatccgttttttacggatggctttttatattgactttttcatcgctaatggttacttttctatcaaacgacaccgaaaggcgatgattattgtgttaaaaaacccaatattataacatccactaatggataatcttacttttagtaagtaggttgcctagtgaaatacaataattccaacattgggccttaatacatattaattagttttaaccctctcactataaaagagttaaatatgactccgcaacctctttaatatcactattacattttgtaacaacattattactttcatcctctgtaccattaacaaataaatgaggattctttaccataaagtctaacaatagatcaataaaccgctgcctttgttctctagccatttcctttctcatagagaaccactcccctatctttcactattcttttaatttcatcttgtctttcacttgatttaaacagctctggattcttcctcaatcccccactatcttagttaatggtagttgcttcagtactctacattttttgctaatcgaggttaaaatccttcaaatcccttgactttaaggtcttaaggttttcatactggcgagtttgttaggaaatctgaatgtagttttgggcatagtaacccctgttgaaaatttacgaggttaaaacgcccactcattcgtactggcttagatgccgaggtctcgaattgagattggttgcattgacgacaaacggttatagcttacaaaactactataccgtccgtatatagcttattggcattgtattgcgccgtacggtgcgtttctcacgcccaacaccgttccctttcgggtgattcgctatataacccgcatatccttgtaataaagcttgtacaagaatagccgtgaatcagtgtcatgatcgccgcttacgagtaactgtttaactccagtcgcacaatcatgtcttattttgaacccccaacactttttgccgctaacgtcttggttatcatcattattaggatttacatcccgtaggcttgttttatgcaaggcttgtgtcctttacctcgcttacgtcagacagttgaatgcaacgacaactgatatccgagtgtactctcctgcattggctacactagaccatctttttaaagtggtagcttccactggaacggaacacaaaaagggcgttctctatatctaaacgccctgtacatttacaagacttctaggtatagaaaacgcccttttatatccgttttattatgtatatgacaatgctagtaattactagttgaaatattcgtagagtaacggtacaataggtgtatctaataagccttgttcgcgaaaacaaggcaaataacttatttgaggttggcgcctcttataagtcgaagtcctgtatggctgtacaggattaagtcattctcgctaaaggttggtagccaatagcatatgagagtggcttttttcatttccgttctattaagttactgctaattttaccgctatgtttctattaaatcaacgaaaatttgataaggttcaccagaaacattaatatttgcgacataaaactcactctccaactttggaggatgg

SEQ ID NO: 4 - DNA, 1381 bp, Artificial Sequence
  source: /note=“Description of Artificial Sequence Synthetic polynucleotide”
ctcaacgcctattccattttcttactctaaaaacttggactgttgggtcgacaatgaagaagacattgatgtaattttgaaaaaatcaacgattttaaacttagatattaacaacgatattatttcagatatttctggttttaattcatctgtaattacttacccagatgctcaattagtacctggtatcaatggcaaagcaattcatttagttaacaatgaatcttctgaagttattgtgcataaagcaatggatattgagtataatgatatgttcaacaatttcactgtgtcattctggttacgtgtaccaaaggtgtcagcttctcatttggaacagtatggtacgaatgaatattcaattatctcatcaatgaagaaacattctttgtctattggttctggttggtcagtctcattaaaaggcaataacttaatttggactcttaaggattctgcaggtgaggtccgtcaaatcacgtttcgtgatttaccagataaattcaatgcttatttagcaaataaatgggtattcattacgattactaacgatcgtttatcatctgctaatttgtatattaacggcgttttaatgggttcagcagaaattactggtcttggtgctattcgtgaagacaataatatcactttgaaattagatcgttgcaataataacaatcaatatgtatctatcgataaatttcgtattttctgtaaagctttaaatcctaaagaaattgagaagctttatacgtcatatttatctatcacttttcttcgtgatttctggggtaatccattacgttatgatacggagtactatttaattccagttgcttcttcttcaaaagacgttcaattaaagaatattactgattatatgtatttaacgaatgcaccgtcatacactaatggcaaattgaatatttactatcgtcgtttatataacggtttgaaatttattattaagcgttatactccgaataatgaaattgattctttcgtaaaatctggtgactttattaagttatatgtttcatacaataataacgaacatatcgtaggttatccaaaagatggtaatgcttttaataaccttgatcgtattttacgtgtaggctataatgctccaggtattccactttataagaagatggaagctgtgaagcttcgtgaccttaaaacttattcagtacaattgaagttgtacgatgataagaatgcatcattaggtttagttggtactcataatggtcaaatcggcaacgatcctaatcgtgatattcttattgcatcaaattggtattttaaccacttaaaagataagattttgggttgtgactggtattttgtgccaacggatgagggttggactcgttaa

SEQ ID NO: 5 - DNA, 735 bp, Artificial Sequence
  source: /note=“Description of Artificial Sequence Synthetic construct”
atggcagaagttcagttagtggaatcaggtggcggtttagtacaaccaggccgttcattacgtttatcttgtgcggcatcaggttttacttttgatgattatgcaatgcattgggttcgtcaagctcctggcaaaggcttagaatgggtgtcagctatcacttggaattctggtcacattgattatgctgattctgtagaaggtcgttttacgatttctcgtgataatgcaaagaattcattatatttacaaatgaactctttacgtgctgaggatacggctgtctattattgcgcaaaagtttcatatttatctactgcttcttcacttgattattggggtcaaggtactttagttactgtctcgagtggtggtggtggctcaggtggtggtggttctggcggtggcggatccgatattcaaatgactcaatctccatcttctttatctgcatcagtgggtgatcgtgtgactattacttgtcgtgcttcacaaggcattcgtaactatttagcatggtatcaacagaaaccaggcaaggcaccaaaattacttatttatgcagcatctactcttcaatcaggtgtgccttcacgtttctctggttcaggttctggtactgattttactttaactatctcttctttacaacctgaagatgtggcaacttactattgtcagcgttataatcgtgcaccttatacttttggtcaaggtacgaaagtagaaattaag

SEQ ID NO: 6 - protein, 174 aa, Bacillus thuringiensis
MSLFHCDFLKDLIGSFVRVNRGGPESQKGTIISVCPDYFVLKNEKGELYYYQLSHLKSITKNAKDCGSSDCDWEDCECADDFEALLLSFKYCWVKINRGGPEKVEGILQDVSCDFVTLIVKEEVILIAIKHIKSVNYNALACGESDESEDSNSKESSSDNSGRARAQRQSSRGR

SEQ ID NO: 7 - protein, 171 aa, Bacillus thuringiensis
VSLFHCDFLKDLIGSFVRVNRGGPESQKGTIISVCQDYFVLKNEKGELYYYQLKHLKSITKNAKECGSSDCEWEDCACAEDFEALLESFKYCWVKINRGGPEKVEGILQDVSCDFVTLIVKEEIILIAIKHIKSVNYNALACGESDESDDSKESSDNSGRARAQRQSSRGR

SEQ ID NO: 8 - protein, 7 aa, Tobacco Etch Virus
ENLYFQG

SEQ ID NO: 9 - protein, 40 aa, Artificial Sequence
  source: /note=“Description of Artificial Sequence Synthetic polypeptide”
RIARLEEKVKTLKAQNSELASTANMLREQVAQLKQKVMNY

SEQ ID NO: 10 - protein, 40 aa, Artificial Sequence
  source: /note=“Description of Artificial Sequence Synthetic polypeptide”
LTDTLQAETDQLEDKKSALQTEIANLLKEKEKLEFILAAY

SEQ ID NO: 11 - DNA, 24 bp, Artificial Sequence
  source: /note=“Description of Artificial Sequence Synthetic primer”
ccatggtgagtttatttcattgtg

SEQ ID NO: 12 - DNA, 20 bp, Artificial Sequence
  source: /note=“Description of Artificial Sequence Synthetic primer”
gcggccgctcttcctctact

SEQ ID NO: 13 - protein, 380 aa, Bacillus subtilis
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTMEDGVVYFNIHHIKSISKNEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSKSGRSSRSKSSSKSSKRSLKSSDYQSSKSGRSSRSKSSSKSSKRSLKSSDYQSSKSSKRSPRSSDYQSSRSPGYSSSIKSSGKQKEDYSYETIVRTIDYHWKRKF

SEQ ID NO: 14 - protein, 171 aa, Bacillus cereus
MSLFHCDFLKDLIGSFVRVNRGGPESQKGTIISVCQDYFVLKNEKGELYYYQLKHLKSITKNAKECGSSDCEWEDCACAEDFEALLESFKYCWVKINRGGPEKVEGILQDVSCDFVTLIVKEEIILIAIKHIKSVNYNALACGESDESDDSKESSDNSGRARAQRQSSRGR

SEQ ID NO: 15 - protein, 149 aa, Bacillus cereus
MENVLCCDQIKCLVGETVKVNLRGPESRVCELVSLGKDYLTLQLPKGELVYYQLKHVKSLVKKVKESKCGDCYSSCFCSDEDTFLDILKDLKYKWVKINRGGPECVEGLLSEVHHGCITLVNGDEVIYVIKSHIKSVSQVVKCKKNEDE

SEQ ID NO: 16 - protein, 173 aa, Artificial Sequence
  source: /note=“Description of Artificial Sequence Synthetic polypeptide”
MSLFHCDFLKDLIGSFVRVNRGGPESQRGTIISVCPDYFVLQNEKGELYYYQLSHLKSITKSAKECGSSDCEWEDCACAEDFEALLESFKYRWVKINRGGPEKVEGILQDVSCDFVTLIVKEEIILIAIKHIKSVNYNALACGESDESDDSSSKESSNNSGRARAQRQSSRGR

SEQ ID NO: 17 - protein, 173 aa, Bacillus thuringiensis
MSLFHCDFLKDLIGSFVRVNRGGPESQKGTIISVCPDYFVLQNEKGELYYYQLKHLKSITKNAKECGSSDCEWEDCACAEDFEALLESFKYCWVKINRGGPEKVEGILQDVSCDFVTLIVKEEIILIAIKHIKSVNYNALACGESDESDDSSSKESSNNSGRARAQRQSSRGR

SEQ ID NO: 18 - protein, 171 aa, Bacillus cereus
MSLFHWDFLKDLIGSFVRVNRGGPESQKGTIISVCPDYFVLQNEKGELYYYQLNHLKSITKNAKECGSSDCEWEDCACAEDFEALLESFKYCWVKINRGGPEKVEGILQDVSCDFVTLIVKEEIILIAIKHIKSVNYNALACGESDESDDSKESSDNSGRARAQRQSSRGR

SEQ ID NO: 19 - protein, 173 aa, Bacillus thuringiensis
MSLFHCDFLKDLIGSFVRVNRGGPESQRGTIISVCPDYFVLQNEKGELYYYQLSHLKSITKSAKECGSSDCEWEDCACAEDFEALLESFKYCWVKINRGGPEKVEGILQDVSCDFVTLIVKEEIILIAIKHIKSVNYNALACGESDESDDSSSKESSNNSGRARAQRQSSRGR

SEQ ID NO: 20 - protein, 174 aa, Bacillus thuringiensis; identical to SEQ ID NO: 6

SEQ ID NO: 21 - protein, 170 aa, Bacillus weihenstephanensis
MSLFHCDFLKDLIGSYVRVNRGGPESQKGIIVAVCSDYFVLKSEKGELSYYQLRHLKSVTKNTKDCEADDYGWLEYEDDFEKLLQSFKYRWVKINRGGPEKIEGILQDVSCDYVTLIVKEEVILISIAHIKSVSYSPVVCGESEESNSESNSESNNSGRTRAQRQSSRGR

SEQ ID NO: 22 - protein, 173 aa, Bacillus thuringiensis; identical to SEQ ID NO: 19

SEQ ID NO: 23 - protein, 173 aa, Artificial Sequence; identical to SEQ ID NO: 16
  source: /note=“Description of Artificial Sequence Synthetic polypeptide”

SEQ ID NO: 24 - protein, 173 aa, Bacillus cereus
MSLFHCDFLKDLIGSFVRVNRGGPESQKGTIISVCPDYFVLQNEKGELYYYQLSHLKSITKSAKECGSSDCEWEDCACAEDFEALLESFKYCWVKINRGGPEKVEGILQDVSCDFVTLIVKEEIILIAIKHIKSVNYNALACGESDESDDSSSKESSNNSGRARAQRQSSRGR

SEQ ID NO: 25 - protein, 177 aa, Bacillus cereus
QSIKAVQNNGTEPKGTLKIGSFVRVNRGGPESQKGTIISVCPDYFVLQNEKGELYYYQLNHLKSITKNAKECGSSDCEWEDCACAEDFEALLESFKYCWVKINRGGPEKVEGILQDVSCDFVTLIVKEEIILIAIKHIKSVNYNALACGESDESDDSKESSDNSGRARAQRQSSRGR

SEQ ID NO: 26 - protein, 165 aa, Artificial Sequence
  source: /note=“Description of Artificial Sequence Synthetic polypeptide”
MKDLIGSFVRVNRGGPESQRGTIISVCPDYFVLQNEKGELYYYQLSHLKSITKSAKECGSSDCEWEDCACAEDFEALLESFKYRWVKINRGGPEKVEGILQDVSCDFVTLIVKEEIILIAIKHIKSVNYNALACGESDESDDSSSKESSNNSGRARAQRQSSRGR

SEQ ID NO: 27 - protein, 397 aa, Geobacillus thermodenitrificans
MEMVAYDMHGASMPLEANRELQSLIGKTVTVYRGGPESKTGKLLDVQSDYLVLFVESEAAMIYYALHHVQSISENVKANSVSSLSSYSDETVMPVRATSFYELLNSLVNEHIQINQGGPESKRGTLLGVNDDHIILFTKDDGIVFYLLRHLKSVRVQMKTEENKAVDAKTPAAELEANAMEDATTEVTGHNDMEAASEAPHIPGGEETHEDQEHDPLPPPVYVKAANVTDLFERLINTWIAINRGGPEAVEGILVPGGSGYCTIVNHDEIIRVNYEHIKSISCGPKGAFQANTMNAGDGKETGQAEAKQEPNQAEREAKKAQKKEEEGVKAKQKEQGDKKKEEKEKEKEQGDKKKEEAKAKKKEQGDKEKEEKEKEKEKEKEKKKEKEKKKEKKKKG

SEQ ID NO: 28 - protein, 255 aa, Artificial Sequence
  source: /note=“Description of Artificial Sequence Synthetic polypeptide”
MTLYGMNDHSMSSLPNDELAPLIGKTVTIYRGGPESKTGKLLDVQSDYLSLMDGSSVIYYALHHVQSITENAKQNSIPSVSDLANPSDTEVPAGFSCFHELLQHLVDAYIQVNQGGPESKQGTLLGVNDDHIVLFTRDDGVVFYHLQHIKSISLPIKREETAPEAGSSANEAANNVSPSSADHVSPSPEDASKHQETLSPPPYVNAQNLSDLLAHLTHTWVAINRGGPEAIEGILCKLAHYFRIPFSNYFGRIRK

SEQ ID NO: 29 - protein, 296 aa, Bacillus amyloliquefaciens
MKRNTKRQRSDDNFLHAMEGKVITVYRGGPESKTGRLADIQSDYIALQVDNKIVYYQWKHVKSVTENTSETVSPAEAAECEKADDFQDLISRMTNRTVQLNQGGPESKRGKLHETGDDFLVLETEDDGIVYFNADHVKSISAEQEEDGQEEERPEFEMADDFHGIFKRLIHKWVSINRGGPEAVEGILVDNSDGHYTLVKDKEVLRIYPFHIKSISEGAKGAAKKEENKDEENKGEKESAEEEKPHEESREEQRTSSKKSKRSSRSVRSSKREKEVSYSYATVLRTIDYRWKHGRK

SEQ ID NO: 30 - protein, 380 aa, Bacillus subtilis
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTNEDGVVYFNIHHIKSISKHEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSKSGRSSRSKSSSKSSKRSLKSSDYQSSKSGRSSRSKSSSKSSKRSLKSSDYQSSKSSKRSPRSSDYQSSRSPGYSSSIKSSGKQKEDYSYETIVRTIDYHWKRKF

SEQ ID NO: 31 - protein, 149 aa, Bacillus cereus
MENVLCCDQIKCLVGETVKVNLRGPESRVGELVSLGKDYLTLQLPHGELVYYQLKHVKSLVKKVKESKCGDCYSSCFCSDEDTFLDILKDLKYKWVKINRGGPECVEGLLSEVHHGCITLVNGDEVIYVIKSHIKSVSQVVKCKKNEDE

SEQ ID NO: 32 - protein, 149 aa, Bacillus cereus
MENVLCCDQIKCLVGETVKVNLRGPESRVGELVSLGKDYLTLQLPHGELVYYHLKHVKSLVKKVKESKCGDCYSSCFCSDEDTFLDILKDLKYKWVKINRGGPECVEGLLSEVHHGCITLVNGDEVIYVVKSHIKSVSQVVKCKKNEDE

SEQ ID NO: 33 - protein, 149 aa, Artificial Sequence
  source: /note=“Description of Artificial Sequence Synthetic polypeptide”
MENVLCCDQIKCLVGETVKVNLRGPESRVGELVSLGKDYLTLQLPHGELVYYQLKHVKSLVKKVKESKCGDCYSSCFCSDEDTFLDILKDLKYKWVKINRGGPECMDGLLSEVHHGCITLVNCDEVIYVINSHIKSVSQVVKCKKNEDE

SEQ ID NO: 34 - protein, 149 aa, Bacillus cereus
MENVLCCDQIKCLVGETVKVNLRGPESRVGELVSLGKDYLTLQLPHGELVYYQLKHVKSLVKKVKESKCGDCYSSCFCSDEDTFLDILKDLKYKWVKINRGGPECVDGLLSEVHHGCITLVNCDEVIYVIKSHIKSVSQVVKCKKNEDE

SEQ ID NO: 35 - protein, 149 aa, Bacillus thuringiensis
MENVLCCDQIKCLVGETVKVNLRGPESRVGELVSLGKDYLTLQLPHGELVYYQLKHVKSLVKKVKESKCGDCYSSCFCSDEDTFLDILKDLKYKWVKINRGGPECVEGLLSEVHHGCITLVNCDEVIYVIKSHIKSVSQVVKCKKNEDE

SEQ ID NO: 36 - protein, 110 aa, Bacillus cereus
MENVLCCDQIKCLVGETVKVNLRGPESRVGELVSLGKDYLTLQLPHGELVYYHLKHVKSLVKKVKESKCGDCYSSCFCSDEDTFLDILKDLKYKWVKINRGGPECVEGLL

SEQ ID NO: 37 - protein, 149 aa, Bacillus thuringiensis
MESILCCEQIKCLVGETVKVNLRGPESRVGVLVSLGRDYLTLQLPLGELVYYQLKHVKSLAKKVKESKCGDSYVSCFCSDDDTFVDVLRDLKYKWVKINRGGPECVEGLLSEVHEECITLVNCDEVIYIINYHIKSVSQVVKCKKNEDE

SEQ ID NO: 38 - protein, 149 aa, Bacillus weihenstephanensis
MESVLCCDQIKGLVGENVKVNLRGPESRVGVLLSLGKDYLTLQLPHGEVVYYQLKHVKSLVKKVKETKCDDYYGSCFYSDEDTFFDVLKDLKYKWIKINRGGPECVEGLLSEVHEECIILVNCDEVIYIINYHIKSVSQIVKCKKNEDE

SEQ ID NO: 39 - protein, 170 aa, Bacillus weihenstephanensis; identical to SEQ ID NO: 21

SEQ ID NO: 40 - protein, 174 aa, Bacillus thuringiensis; identical to SEQ ID NO: 6

SEQ ID NO: 41 - protein, 171 aa, Bacillus cereus; identical to SEQ ID NO: 14

SEQ ID NO: 42 - protein, 171 aa, Bacillus cereus; identical to SEQ ID NO: 18

SEQ ID NO: 43 - protein, 173 aa, Bacillus thuringiensis; identical to SEQ ID NO: 17

SEQ ID NO: 44 - protein, 171 aa, Bacillus thuringiensis; identical to SEQ ID NO: 14

SEQ ID NO: 45 - protein, 113 aa, Bacillus thuringiensis
SLCDLKLIGSFVRVNGGPESRKGTLISVDYVLQEGELVYYQLKHLKSITKKEDCDADDFLLSLKYKWVKINRGGPEVEGILDVSDFVTLIEEVIIHIKSVAGSDESSGRASRR

SEQ ID NO: 46 - protein, 11 aa, Bacillus thuringiensis
MDNNPNINECI

SEQ ID NO: 47 - protein, 11 aa, Bacillus thuringiensis
MDNNPKIQECI

SEQ ID NO: 48 - protein, 11 aa, Bacillus thuringiensis
MEIVNNQNQCV

SEQ ID NO: 49 - protein, 11 aa, Bacillus thuringiensis
MEIMNNQNQCV

SEQ ID NO: 50 - protein, 10 aa, Bacillus thuringiensis
MEINNQKQCI

SEQ ID NO: 51 - protein, 10 aa, Bacillus thuringiensis
MEENNQNQCI

SEQ ID NO: 52 - protein, 10 aa, Bacillus thuringiensis
MENNIENQCI

SEQ ID NO: 53 - protein, 10 aa, Bacillus thuringiensis
MDINHQNQCI

SEQ ID NO: 54 - protein, 17 aa, Artificial Sequence
  source: /note=“Description of Artificial Sequence Synthetic peptide”
LEDAEFRHDSGYEVHHQ

SEQ ID NO: 55 - DNA, 1016 bp, Homo sapiens
ccatggcttctcctgcacctccagcatgtgatcttcgtgtgttatctaaacttttgcgtgattctcatgtacttcattctcgtctttctcaatgtcctgaagttcatcctttacctactcctgtacttttgcctgctgtcgatttttcattaggcgaatggaaaacgcaaatggaggaaacgaaggctcaggatattttaggtgctgtcacgttattattagaaggcgttatggcagcacgtggtcaattaggcccaacttgtttatcttcattgttgggtcaattatctggtcaagtacgtttacttttaggcgctttacaatctttattaggtactcaattaccgcctcagggtcgtactactgcgcataaagatccaaatgctatttttttatcattccagcatttattacgtggtaaagtgcgttttttgatgttagtaggcggttcaactttatgtgtccgtcgtgcaccaccgacgactgcagtaccatctcgtacttctcttgttttaactcttaacgagttacctaatcgtacttctggcttattagaaactaatttcactgcttctgctcgtacgacgggttctggtttacttaaatggcaacaaggttttcgtgctaaaattccgggtttattgaatcaaacgtctcgttcattggatcaaattccgggctacttaaatcgtattcacgagttattaaatggtacgcgtggtttatttccaggcccatctcgtcgtactttaggtgctcctgatatttcatctggtacttctgacactggctctttaccacctaacttacaaccaggttattcaccatctccaacgcatccaccaacgggtcaatatactttattcccattacctccgactttaccgactccagtagttcaattgcatccacttttacctgatccatctgcaccaacgccgacgcctacttctccgcttttaaatacttcatatacgcattctcaaaacttgtctcaagagggtcgtgcggccgca